(19) United States
(12) Patent Application Publication
(10) Pub. No.: US 2004/0002154 A1
(43) Pub. Date: Jan. 1, 2004

(54) Methods for preparing libraries of unique tags and related screening methods

(76) Inventor: Bernhard O. Palsson, La Jolla, CA (US)

Correspondence Address: MCDERMOTT, WILL & EMERY, 7th Floor, 4370 La Jolla Village Drive., San Diego, CA, 92122, US

(21) Appl. No.: 10/418,754

(22) Filed: Apr. 18, 2003

Related U.S. Application Data
(60) Provisional application No. 60/374,005, filed on Apr. 19, 2002.

(51) Int. Cl. G01N 33/53; C12N 1/00; C12N 15/01

(52) U.S. Cl. 435/446; 435/317.1; 435/7.1

(57) ABSTRACT

The invention provides a method for selecting a population of non-cellular physical entities. The method involves applying energy to one or more non-cellular physical entities having selected parameter signatures, each physical entity located at specific coordinates in a domain and contained within a population of physical entities, thereby altering a property of the one or more physical entities, wherein the alteration renders the one or more physical entities separable from other members of the population of physical entities. The invention also provides a methods for preparing a population of uniquely tagged non-cellular physical entities, and methods for preparing a population of uniquely tagged probes. The invention further provides a method for simultaneously detecting a plurality of analytes using the uniquely tagged probes.

This application claims benefit of the filing date of U.S. Provisional Application No. 60/374,005, filed Apr. 19, 2002, which is incorporated herein by reference.

### BACKGROUND OF THE INVENTION

This invention relates generally to the field of genome and proteome analysis and, more specifically to methods for preparing populations of unique tags useful for genome and proteome analysis.

A variety of molecular assays can be used to identify a single analyte, such as a nucleic acid or protein, in a biological sample. These assays can be used, for example, to detect a known mutation in a gene, an infectious agent, or a protein associated with a disease. The increasing need to identify multiple analytes in a single sample has become increasingly apparent in many branches of medicine. For example, it can be desirable to analyze a single sample for the presence of several infectious agents at once, for several genes that are involved in a particular disease, or for several genes that are involved in different diseases.

The full sequencing of the human genome has facilitated methods for comparing all of the genes between different cells or individuals. Different individuals are known to contain single base pair changes, called single nucleotide polymorphisms (SNPs), throughout their genomes. It is believed that there will be about one polymorphism per 1,000 bases, resulting in a large number of differences between individuals. These single nucleotide differences between individuals can result in a wide variety of physiological consequences. For example, the presence of different SNPs in cytochrome P450 genes can predict the ability or inability to metabolize certain drugs. Screening individuals for the presence of multiple SNPs could be used to predict how an individual will respond to a particular drug or treatment. In addition, a collection of SNPs can define a unique genotype to every individual. Estimates of the number of SNPs needed to get a unique human genotype vary but fall in the range of 30,000 to 40,000 Thus, there is a great need to perform a large number of simultaneous assays of SNPs or any other unique polymorphic sequences.

Proteomics is the study of proteins expressed in a cell. Although more complex than genomics, proteomic analysis can give a more accurate picture of the state of a cell than genomic analysis. For example, the level of mRNA transcribed from a gene does not always correlate to the level of expressed protein. Therefore, analysis of gene expression alone does not always give an accurate picture of the amount of protein derived from a gene of interest. In addition, many proteins are post-translationally modified and these modifications are often important for activity. The type and level of modification of a protein can not be accurately predicted using genome analysis. Therefore, it is important to study a cell in terms of the proteins that are present. For example, it can be desirable to identify and quantitate all proteins present in a cell from an individual and compare the profile with other cells from the same or different individuals.

Assays for the detection of single proteins using antibody-based assays are available. However, analysis of several proteins simultaneously in the same sample can be more difficult. Two-dimensional gel electrophoresis has been used to study the protein content of a cell. This technique requires an individual gel for each sample and sophisticated software to compare the pattern of protein spots between gels. In addition, it is difficult to detect low abundance proteins using this method and several proteins, such as membrane proteins or proteins of very low or high molecular weight, are not amenable to the analysis.

Thus, there exists a need for methods to identify a plurality of analytes, including nucleic acids and proteins, quickly and with high sensitivity, high accuracy, and a large dynamic range. The present invention satisfies this need and provides related advantages as well.

### SUMMARY OF THE INVENTION

The invention provides a method for selecting a population of non-cellular physical entities. The method involves applying energy to one or more non-cellular physical entities having selected parameter signatures, each physical entity located at specific coordinates in a domain and contained within a population of physical entities, thereby altering a property of the one or more physical entities, wherein the alteration renders the one or more physical entities separable from other members of the population of physical entities.

The invention also provides a method for preparing a population of uniquely tagged non-cellular physical entities. The method involves (a) contacting a population of non-cellular physical entities with a chemical agent; (b) applying energy to one or more targeted physical entities, the energy capable of inducing attachment of the chemical agent to a targeted physical entity; (c) separating unattached chemical agent from chemical agent attached to the one or more targeted physical entities, and (d) repeating steps (a), (b) and (c) using a distinct chemical agent to produce a population of uniquely tagged non-cellular physical entities.

The invention provides another method for preparing a population of uniquely tagged non-cellular physical entities. The method involves (a) associating a population of physical entities with two or more reaction spaces on a domain, each reaction space containing a different chemical agent, and (b) applying energy to a targeted physical entity in each of one or more reaction spaces, the energy capable of inducing attachment of a chemical agent to the physical entity, thereby generating a population of uniquely tagged physical entities.

The invention further provides a method for preparing a population of uniquely tagged probes. The method involves (a) contacting a population of uniquely tagged non-cellular physical entities with a target moiety;(b) applying energy to one or more targeted uniquely tagged physical entities, the energy capable of inducing attachment of-the target moiety to a targeted physical entity; (c) separating unattached target moiety from target moiety attached to the one or more targeted uniquely tagged physical entities, and (d) repeating steps (a), (b) and (c) using a distinct target moiety to label another member of the population of physical entities, thereby generating a population of uniquely tagged probes.

The invention provides another method for preparing a population of uniquely tagged probes. The method involves (a) associating a population of uniquely tagged physical entities with two or more reaction spaces on a domain, each reaction space containing a different target moiety, and (b) applying energy to a targeted uniquely tagged physical entity in each of one or more reaction spaces, the energy capable of inducing attachment of a target moiety to the physical entity, thereby generating a population of uniquely tagged probes.

The invention provides a method for simultaneously detecting a plurality of analytes. The method involves (a) contacting a population of uniquely tagged probes prepared according to the claimed methods for preparing a population of uniquely tagged probes, and (b) detecting an interaction between one or more uniquely tagged probe and a cognate binding partner.

### BRIEF DESCRIPTION OF DRAWINGS

FIG. 1 is a perspective view of one of a physical entity targeting apparatus and illustrates the outer design of the housing and display.

FIG. 2 is a perspective view of a physical entity targeting apparatus with the outer housing removed and the inner components illustrated.

FIG. 3 is a block diagram of the optical subassembly design for a physical entity targeting apparatus.

FIG. 4 is a front view of the relative focal planar regions achieved at stepped Z-levels by the CCD array.

FIG. 5 is a perspective view of a domain showing how the three-dimensional image processor module assembles the images captured by the CCD array at stepped Z-levels.

FIG. 6 is a bottom view of a domain containing physical entities illustrating the quadrants as seen by the CCD array. Each rectangular quadrant represents an image captured by a single camera focused at its respective Z-level.

FIG. 7 is a block diagram of the optical subassembly that illustrates the interrelation of the CCD array with the physical entity targeting apparatus.

FIG. 8 is a perspective view of an optical subassembly of a physical entity targeting apparatus.

FIG. 9 is a side view of an optical subassembly that illustrates the arrangement of the scanning lens and the movable stage.

FIG. 10 is a bottom perspective view of an optical subassembly.

FIG. 11 is a top perspective view of the movable stage of the physical entity targeting apparatus.

FIG. 12 is a diagram depicting a uniquely tagged physical entity on a domain.

### DETAILED DESCRIPTION OF THE INVENTION

This invention is directed to methods for preparing populations of unique tags, which can be used to label probes for a variety of molecular diagnostic, screening and analyte detection methods, including SNP detection, mRNA expression profiling, proteomic profiling, drug screening, and target identification. The methods can be used to prepare a population of tens, hundreds, thousands or tens of thousands of unique tags.

In one embodiment, the invention is directed to a method for labeling a population of physical entities to obtain a large population of unique tags. The method involves selectively labeling members of a diverse population of physical entities, such as beads, by applying energy to each target physical entity. Rather than labeling each physical entity in a separate vessel or well and later combining them to generate a population of unique tags, the method involves labeling each unique physical entity while it is present in a population of other untagged physical entities or unique tags. The uniquely tagged physical entities, or “unique tags” can be used to label probes for applications requiring a large population of distinguishable uniquely labeled probes.

In another embodiment, the invention is directed to a method for synthesizing a large population of unique tagged probes. The method involves selectively labeling members of a population of uniquely tagged physical entities, such as uniquely tagged beads, by applying energy to the physical entities to either attach target moieties to the physical entities or to synthesize target moieties on the physical entities. The uniquely tagged probes can be used in a variety of applications involving detection of multiple target analytes, including diagnostic and prognostic tests, nucleic acid sequencing, and genome and proteome analysis.

In certain embodiments, the methods for preparing unique tags involve using an imaging system to detect, synthesize and manipulate physical entities, such as beads and other particles. An optical scanner is used to rapidly image physical entities contained within a domain, such as a solid surface or three dimensional area. Because the physical entities are distinguished from each other by specific signatures, an optical scanner that can detect the particular characteristic of a population of physical entities can locate one, many, hundreds, or even thousands of different physical entities within a domain. One or more focused energy sources, such as a laser or electron beam, are used to further manipulate the properties of a physical entity, for example by inducing a chemical reaction on a physical entity, altering a property of a physical entity or destroying a physical entity.

As used herein, the term “non-cellular physical entity” is intended to mean a particle that is not a cell. Exemplary non-cellular physical entities include beads, such as polymerized beads, partially polymerized beads and non-polymer beads, which can be permeable, semi-permeable, solid and hollow; microcapsules; artificial membrane structures, such as micelles and vesicles; and nanotubes and other particle geometries. A physical entity generally exhibits a high degree of uniformity with respect to size, or to a set of different size classes such that the high degree of uniformity within each class size is sufficient to permit the use of particle size as a parameter signature. A physical entity generally is approximately spherical, although any particle geometry can be employed. A physical entity can be made of a variety of materials, such as polystyrene, latex, carbohydrate-based polymers, polyaliphatic alcohols, poly(vinyl) polymers, polyacrylic acids, polyorganic acids, polyamino acids, co-polymers, block co-polymers, tert-polymers, npolyethers, naturally occurring polymers, polyimids, branched polymers, polyaldhydes, cyclo-polymers and mixtures thereof.

As used herein, the term “specific signature,” when used in reference to a physical entity is intended to mean a detectable characteristic, or combination of detectable characteristics, of a physical entity that distinguishes it from other physical entities in a population of physical entities. A physical entity having a specific signature is referred to herein as a “uniquely tagged physical entity.” A physical entity can have a variety of characteristics that define or contribute to its specific signature. As used herein, the term “parameter signature” is intended to mean a characteristic of a physical entity that defines or is an attribute of its specific signature. A parameter signature can be, for example, size; shape; fluorescence lifetime, fluorescence polarization, fluorescence absorption or fluorescence emission of a compound contained within or on a particle; positron emission, alpha, beta or gamma radiation emission; hydrophobicity; hydophilicity; chemical reactivity; density, concentration, or dye type, contained within or on a particle, or any other physical property of a particle that can be detected. A dye type can include a dye that absorbs or emits a particular wavelength or color of visible light or fluorescence. A parameter signature can be described quantitatively by a discreet value or by a range of values. When a specific signature is defined by two or more parameter signatures, the two or more characteristics can be described quantitatively by two or more values or expressions, a ratio of the two or more values or expressions or other mathematical manipulation of the two or more values or expressions, such as a single value or expression that describes a combination of parameter signatures that define a specific signature. In one embodiment of the methods of the invention, an optical device can simultaneously detect two or more characteristics of a physical entity, which together define the specific signature of the physical entity.

As used herein, the term “specific location” is intended to mean the two-dimensional or three-dimensional coordinates of a physical entity with respect to the domain in which the physical entity is contained, or a portion of the domain.

As used herein, the term “domain” is intended to mean an area, including a surface or three-dimensional space, for which an image can be obtained. Exemplary domains include slides, plates, tubes, vessels, arrays, particles and other configurations of matter that provide a surface or three-dimensional space that can contain a physical entity.

As used herein, the term “reaction space” is intended to mean a portion or area of a domain that lacks liquid communication with other portions or areas of the domain. Exemplary reaction spaces include sample wells, array locations, tubes or other vessel that prevents fluid flow between two samples, such as two samples containing populations of entities, or containing different individual entities. A reaction space is used to maintain separation of components within two or more samples.

As used herein, the term “attachment” is intended to mean linkage of a chemical reagent or moiety to a substrate, which can be, for example, a moiety contained on a physical entity or another chemical reagent or moiety attached to a physical entity. The term attachment includes linkages mediated by a variety of chemical reactions, including those induced by energy, such as light (photoattachment) and heat, and a chemical or enzyme-induced reaction.

As used herein, the term “uniquely tagged entity” is intended to mean a physical entity that is distinguishable from other entities within a population of entities. As used herein, the term “uniquely tagged probe” is intended to mean a uniquely tagged entity linked to a target moiety. A target moiety is a molecular entity that interacts with a binding partner to form a specific binding pair. The affinity of a target moiety for a cognate binding partner will generally be greater than about 10⁻⁵ M, for example greater than 10⁻⁶ M, including greater than about 10⁻⁸ M and greater than about 10⁻⁹ M. A target moiety can have a variety of molecular structures. For example, a target moiety can be a naturally occurring macromolecule, such an antibody, polypeptide, nucleic acid, carbohydrate, or lipid, or a modification thereof. A target moiety can also be a partially or completely synthetic derivative, analog or mimetic of such a macromolecule, or a small organic molecule. A target moiety can be linked to a physical entity by covalent or non-covalent interaction, absorption, dissolution, surface adsorption, and the like. A target moiety also can be contained within a physical entity that is a bead, microcapsule, micelle, vesicle, or other hollow or porous structure that can envelope, absorb, or otherwise contain a target. A binding partner that associates with a target moiety can be, for example, an analyte, receptor, ligand, antibody, antigen, nucleotide sequence, polypeptide and the like.

As used herein, the term “energy” is intended to mean an emission from a laser, electron beam, or high-powered broad band light source, such as a an arc lamp or quartz halogen lamp, which can be diffuse or concentrated into a beam sufficiently small to target a physical entity. As used herein, the term “controlled energy source” is intended to mean an emission from a laser, electron beam, or high-powered broad band light source, such as a an arc lamp or quartz halogen lamp that is concentrated into a beam sufficiently small to target a physical entity. Exemplary energy sources include arc lamps, such as mercury arc lamps and xenon arc lamps, and lasers, such as argon ion or krypton ion lasers, helium neon lasers, helium cadmium lasers, dye lasers, such as rhodamine 6G lasers, YAG lasers and diode lasers. As used herein, the term “focal planar region” is intended to mean a viewed region in three-dimensional space that is elongated in two dimensions and substantially confined between two parallel planes that are orthogonal to the direction of view. The viewed region can be a slice or section of a domain or portion of such a slice or section. Thus, a focal planar region of a domain can be used to produce a sectional image of the domain. The midplane of a focal planar region is intended to mean the plane that is parallel to and midway between the two parallel planes that confine the focal planar region;

The invention provides a method for selecting a population of non-cellular physical entities. The method involves applying energy to one or more non-cellular physical entities having selected parameter signatures, each physical entity located at specific coordinates in a domain and contained within a population of physical entities, thereby altering a property of the one or more physical entities, wherein the alteration renders the one or more physical entities separable from other members of the population of physical entities.

A variety of non-cellular particles can be physical entities useful in the methods of the invention. For example, beads of various sizes, compositions and geometries; micelles; vesicles, monolayer and multilayer assemblies; quantum dots and other microscopic particles, and the like, which have or can be made to have different parameter signatures can be used. Once method for imparting different parameter signatures to physical entities involves incorporating dyes and combinations of dyes into the particles. Such dyes can impart color, fluorescence of another detectable parameter signature to a physical entity. Exemplary fluorescent and chromogenic moieties include Alexa Fluor Dyes, BODIPY fluorophores, fluorescein, Oregon Green, eosins and erythrosins, Rhodamine Green, tetramethylrhodamine, Lissamine Rhodamine B and Rhodamine Red-X Dyes, Cascade Blue dye, coumarin derivatives, naphthalenes, including dansyl choloride. Methods for preparing dye-containing beads are described, for example in WO 99/52708, the entirety of which is incorporated herein by reference. Micelles and other particles also can be prepared to contain dyes.

Those skilled in the art will know how to select an appropriate dye, for example, based on the emission, absorption and hydrophobic/hydrophilic properties desired, photostability and quantum yield. When more than one dye is used in a physical entity, the selected dyes can have similar or overlapping excitation spectra but different emission spectra, such that the dyes are spectrally distinct. When differentiation between two or more dyes is accomplished by visual inspection, the two or more dyes generally have emission wavelengths of perceptibily different colors to enhance visual discrimination. When differentiation between two or more dyes is accomplished by instrumentation, a variety of filters and diffraction gratings are commercially available to allow the respective emission maxima to be independently detected. When two or more dyes are selected that possess relatively small differences in emission maxima, instrumental discrimination can be enhanced by ensuring that the emission spectra of the two or more dyes have similar integrated amplitudes and similar emission peak widths and that the instrumental system's optical throughput will be equivalent across the emission peak widths of the respective two dyes.

The method can be used to select a homogeneous or heterogeneous populations of non-cellular physical entities. A homogenous population of non-cellular physical entities are a group of physical entities in which each member of the group has a parameter signature that is substantially the same as that of other members of the group. A heterogeneous population of non-cellular physical entities are a group of physical entities in which members have different signature parameters. In one embodiment, the method is used to select a population of physical entities that each have a distinct signature parameter. To select a homogenous or heterogenous population of non-cellular physical entities, those entities to be retained in the population are referred to as physical entities having a “desired signature parameter,” whereas physical entities to be excluded from a population are referred to as physical entities having an “undesired signature parameter.” The method also is applicable to selecting homogeneous or heterogenous populations of non-cellular physical entities that based on two or more signature parameters, which can be a “specific signature.”

The methods of the invention involve altering a physical entity such that it becomes separable from other members of a population of physical entities. Exemplary properties of a physical entity that can be altered to render the physical entity separable include mass, such that reducing or increasing the mass of a particle can render it separable. For example, destroying the physical entity by reducing its mass renders the physical entity separable from other physical entities in-a population. When a physical entity is rendered separable from a population of physical entities by its destruction or disintegration, a physical separation step is not required to obtain a population of physical entities having desired signature parameters, although a physical—separation step can be performed to remove unwanted residual material. Adding mass to a physical entity also can render it separable from other physical entities in a population, for example, when a label such as biotin or magnetic compound is attached to the physical entity. Another alteration of a physical entity that renders it separable is attachment of the physical entity to the domain. Attachment of a physical entity to the domain can be used to either retain the physical entity within a population or to discard it from the population.

Attachment of a physical entity to a domain can be accomplished using a variety of well-known non-covalent and covalent interactions and chemical reactions, including those induced by energy, such as light (photoattachment) and heat, or chemical or enzyme-induced reactions. In one embodiment, attachment of a physical entity to a domain is performed by applying energy to a targeted physical entity.

For example, the methods can be used to select a population of physical entities by attaching physical entities having a desired property to a domain, leaving undesired physical entities unbound to the domain, washing away the undesired physical entities and optionally unattaching the desired physical entities; by attaching undesired physical entities to a domain, leaving desired physical entities unbound to the domain and collecting the unbound physical entities; by destroying unwanted physical entities, leaving desired physical entities intact and collecting the intact physical entities; by altering a property of a desired or undesired physical entity so that the physical entity becomes separable or inseparable, and collecting the desired population of bound or unbound physical entities.

The methods of the invention involve applying energy, for example from a pulse of a controlled energy source, to a physical entity at particular coordinates. Upon application of energy, a physical entity can be altered in a variety of ways. For example, an energy beam can be used to photomechanically disrupt, photodissociate, photoablate, rearrange, isomerize, dimerize, eliminate or add small molecule, or undergo energy transfer or electron transfer with another molecule. For example, a sufficient amount of energy can be supplied to specifically activate a photosensitive substance, including a caged compound that acts locally to react with a physical entity but does not react with other physical entities, to induce a physical or chemical change on the physical entity, including destroying the physical entity. Chemical changes induced by light are described, for example, in Horspool, Synthetic Organic Photochemistry, (Plenum Press, New York and London)(1984).

The energy delivered by the pulse of a controlled every source can be limited, for example, to at most 2, 1.5, 1, 0.7, 0.5, 0.3, 0.2, 0.1, 0.05, 0.02, 0.01 or even 0.005 μJ/μm². The pulse from a controlled energy source can have a maximum duration, for example, of approximately 100 milliseconds, 1 millisecond, 10 microseconds, 1 microsecond, 100 nanoseconds, 10 nanoseconds, 1 nanosecond or less than 1 nanosecond.

The methods of the invention can involve separating the one or more altered physical entities from the population of physical entities. Separation of a population of physical entities having desired signature parameters can be performed using a variety of well-known methods, including washing, binding of physical entities to a selective ligand, such as avidin, biotin, metal binding materials and other affinity binding materials, mechanical separation, filtration, electrophoretic methods, magnetic separation methods and the like. Separation of a physical entity from a population also can be achieved by attaching the physical entity to a domain and removing unattached physical entities.

The methods of the invention for selecting a population of non-cellular physical entities can involve altering properties of a few, many, hundreds, thousands and tens of thousands of physical entities. Therefore, energy can be applied to 10 or more, 100 or more, 1000 or more, 10⁴ or more, 10⁵ or more, 10⁶ or more, 10⁷ or more, 10⁸ or more, or 10⁹ or more, non-cellular physical entities having selected parameter signatures.

The methods of the invention involve determining the coordinates of a physical entity in a domain. Coordinates can be determined using a variety of well-known methods. For example, to determine coordinates of a physical entity in two dimensions, an image of a population of physical entities can be captured, a particular physical entity within the population can be identified, and coordinates can be assigned to that physical entity based on its two-dimensional location in a field. The coordinates of a physical entity also can be determined in three dimensional space. For example, to determine coordinates of a physical entity in three-dimensional space by obtaining a plurality of nonidentical two-dimensional sectional representations of the domain, in which the physical entity is discernable in at least one of the sectional representations; combining the plurality of sectional two-dimensional representations to produce a three-dimensional representation of the domain; locating the physical entity in three dimensions based on the three-dimensional representation, and assigning coordinates to the physical entity. Coordinates of one or more physical entities can be indexed, for example, in a database, computer memory or machine capable of storing such data.

The invention provides a method for preparing a population of uniquely tagged non-cellular physical entities. The method involves (a) contacting a population of non-cellular physical entities with a chemical agent; (b) applying energy to one or more targeted physical entities, the energy capable of inducing attachment of the chemical agent to a targeted physical entity; (c) separating unattached chemical agent from chemical agent attached to the one or more targeted physical entities, and (d) repeating steps (a), (b) and (c) using a distinct chemical agent to produce a population of uniquely tagged non-cellular physical entities. Step (d) optionally can involve repeating steps (a), (b) and (c) 10 or more times, 100 or more times, 1000 or more times, 10⁴ or more times, 10⁵ or more times, 10⁶ or more times, 10⁷ or more times, either simultaneously or consecutively, for example, by using one or more controlled energy sources.

A variety of chemical agents can be attached to a physical entity, depending on the desired population of physical entities. For example, a chemical agent can be a unique tag that imparts a unique specific signature to a physical entity. Such a chemical agent can be any moiety that imparts a parameter signature to a physical entity, or any moiety that is a building block used to synthesize a tag that imparts a parameter signature to a physical entity. Exemplary chemical agents that can impart a parameter signature to a physical entity include a dye or dye-containing particle, polynucleotide, polypeptide, radioactive substance or other detectable moiety that can impart a parameter signature to a physical entity. A chemical agent also can be a building block employed in the process of synthesizing a unique tag on a physical entity. For example, two or more fluorescent dyes, fluorescent dye-containing particles, such as nanoparticles, radioactive moieties or other moieties having detectable physical properties, can be used to impart a specific parameter signature. Further, a chemical agent can be a building block employed in the process of synthesizing a target moiety on a physical entity. For example, a chemical agent can be one of multiple nucleotides or amino acids used to synthesize a polynucleotide or polypeptide that functions as a parameter signature or target moiety.

The method for preparing a population of uniquely tagged non-cellular physical entities also can be used to prepare a population of uniquely tagged probes. To prepare a population of uniquely tagged probes, a population of physical entities having distinct specific signatures are used, and target moieties are either attached to the physical entities (when a chemical agent comprises a target moiety), or synthesized on the physical entities (when a chemical agent comprises a moiety that functions as a building block of a target moiety).

In one approach, an uniquely tagged physical entity can be constructed sequentially from a single or several monomeric phosphoramidite building blocks (one containing a dye residue), which are chosen to generate tags with unique sequences. The uniquely identifying tag is thus composed of monomeric units of variable sequence bridged by phosphate linkers. In one approach, an uniquely tagged physical entity can be constructed sequentially from a single or several monomeric phosphoramidite building blocks (one containing a dye residue), which are chosen to generate tags with unique sequences. The uniquely identifying tag is thus composed of monomeric units of variable sequence bridged by phosphate linkers.

The invention provides another method for preparing a population of uniquely tagged probes. The method involves (a) contacting a population of uniquely tagged non-cellular physical entities with a target moiety; (b) applying energy to one or more targeted uniquely tagged physical entities, the energy capable of inducing attachment of the target moiety to a targeted physical entity; (c) separating unattached target moiety from target moiety attached to the one or more targeted uniquely tagged physical entities, and (d) repeating steps (a), (b) and (c) using a distinct target moiety to label another member of the population of physical entities, thereby generating a population of uniquely tagged probes.

A variety of molecular entities can serve as a target moiety. For example, a target moiety can be a small structure, such as an organic compound, lipid, carbohydrate, short amino acid sequence, oligonucleotide, or other small structure capable of selectively interacting with a binding partner. A target moiety also can be a large structure, such as one or more polynucleotide and polypeptide, for example an antibody, antigen, ligand and receptor.

A target moiety can be attached to a physical entity using a chemistry suitable for the particular target moiety and physical entity. For example, application of energy to a physical entity in the presence of another molecule can be used to rearrange, isomerize, dimerize, or otherwise link the molecule to the physical entity. As described above in relation attaching a chemical agent to a physical entity, a target moiety can be synthesized on a physical entity by a series of reactions in which multiple chemical reagents serve as building blocks to produce a target moiety. For example, a uniquely tagged physical entity can be labeled with a nucleic acid sequence target moiety by multiple additions of particular nucleotide residues.

One or more members of a population of physical entities, such as a targeted physical entity or targeted uniquely tagged physical entity, can be attached to a specific location on a domain. Attachment of a physical entity to a domain can be specific, for example by attaching one or more targeted physical entities, or non-specific, for example by attaching the population of physical entities, or a portion thereof. A variety of methods can be used for specific and non-specific attachment of a physical entity to a domain, and the method selected will depend on the properties of the physical entities and the domain. Exemplary methods for attaching a physical entity to a domain include applying energy to a physical entity to attach it to a domain, applying a magnetic field to a physical entity having appropriate magnetic properties to attach it to a domain and using a domain coated with a physical entity-specific ligand to attach a physical entity to a domain.

The invention provides another method for preparing a population of uniquely tagged non-cellular physical entities. The method involves (a) associating a population of physical entities with two or more reaction spaces on a domain, each reaction space containing a different chemical agent, and (b) applying energy to a targeted physical entity in each of one or more reaction spaces, the energy capable of inducing attachment of a chemical agent to the physical entity, thereby generating a population of uniquely tagged physical entities. Steps (a) and (b) optionally can be repeated one or more times, each time using a distinct chemical agent in each reaction space, the chemical agent capable of attachment to a physical entity or chemical agent attached to a physical entity.

The method involves associating a population of physical entities with two or more reaction spaces on a domain. A population of physical entities can be associated with a reaction space by being physically confined to the reaction space, either because the physical entity is attached to the reaction space or because the reaction space is physically isolated by absence of liquid contact with other reaction spaces. For example, a population of physical entities, which can be a diverse population of physical entities having different parameter signatures or different specific signatures, can be distributed among multiple wells of a multi-well plate, slide or chamber. A particular target physical entity can be selected from each well and attached to a chemical agent by applying energy to the targeted physical entity. Because the targeted physical entities are selected such that each one has a unique parameter signature, such as a different size, fluorescence absorption, fluorescence emission or other property, or a specific signature resulting from a combination of two or more parameter signatures, the resulting population of tagged physical entities contains physical entities that are distinct from each other.

The method can be used to prepare a population of uniquely tagged probes. In one embodiment, the method involves (a) associating a population of uniquely tagged, physical entities with two or more reaction spaces on a domain, each reaction space containing a different target moiety, and (b) applying energy to a targeted uniquely tagged physical entity in each of one or more reaction spaces, the energy capable of inducing attachment of a target moiety to the physical entity, thereby generating a population of uniquely tagged probes.

Once a chemical entity has been tagged with a chemical reagent, unattached chemical agent can be separated from chemical agent attached to the physical entity. A separation step can be useful when attachment of a physical entity to a second chemical agent is desired.

The invention provides a method for simultaneously detecting a plurality of analytes. The method involves contacting a population of uniquely tagged probes prepared using the claimed methods for preparing a population of uniquely tagged probes, with a sample and (b) detecting an interaction between one or more uniquely tagged probes and a cognate binding partner.

An interaction between one or more uniquely tagged probes and a cognate binding partner can be detected by a change in a parameter signature of a physical entity, such as a change in fluorescence lifetime, fluorescence polarization, fluorescence absorption or fluorescence emission of a compound contained within or on a physical entity, density, size or shape.

In the methods for simultaneously detecting a plurality of analytes, two or more physical entities having a common specific signature can be employed in the same assay, so long as the physical entities can be distinguished based on another parameter, such as coordinates on a domain, location on an array or presence in a particular sample.

The methods of the invention involve detecting a parameter signature. A parameter signature can be detected using an instrument appropriate for the particular parameter signature. For example, parameter signatures of size and shape can be detected using an imaging system suitable for the physical properties of the particles or a flow cytometric method; light absorption or emission can be detected using a spectrophotometer, fluorometer, luminometer microscope, fluorescence scanner, flow cytometer, confocal microscope, scanning microscope, epifluoresis detector, digital camera, video camera, photgraphic film, visual inspection, photodiode, quantum counter, photomultiplier tube, capillary electrophoresis detector, or any combination thereof; fluorescence absorption, emission, energy transfer, lifetime and polarization can be detected using a fluorometer, radioactivity can be measured using a gamma counter, beta counter, and scintillation counter.

The methods of the invention involve determining the coordinates of a targeted physical entity. A variety of methods can be used to determine X,Y coordinates or three-dimensional coordinates of a particle in or on a domain. In one embodiment, an imaging system is used to determine the coordinates of a physical entity by (a) capturing an image of the population of physical entities; (b) identifying a targeted physical entity in the image and (c) assigning coordinates to the targeted physical entity. In another embodiment, an imaging system is used to determine the coordinates of a physical entity in or on a domain by (a) obtaining a plurality of nonidentical two-dimensional sectional representations a domain containing physical entities, in which the targeted physical entity is discernable in at least one of the sectional representations; (b) combining the plurality of sectional two-dimensional representations to produce a three-dimensional representation of the domain; (c) locating the targeted physical entity in three dimensions based on the three-dimensional representation, and (d) assigning coordinates to the targeted physical entity. Once the coordinates of one or more physical entities have been determined, the coordinates can be indexed in a database.

A variety of apparatus and instrument configurations can be used to determine the coordinates of a physical entity and to apply energy to a targeted physical entity. Exemplary apparatus useful in the methods of the invention are described in U.S. Pat. No. 5,874,266, and herein below.

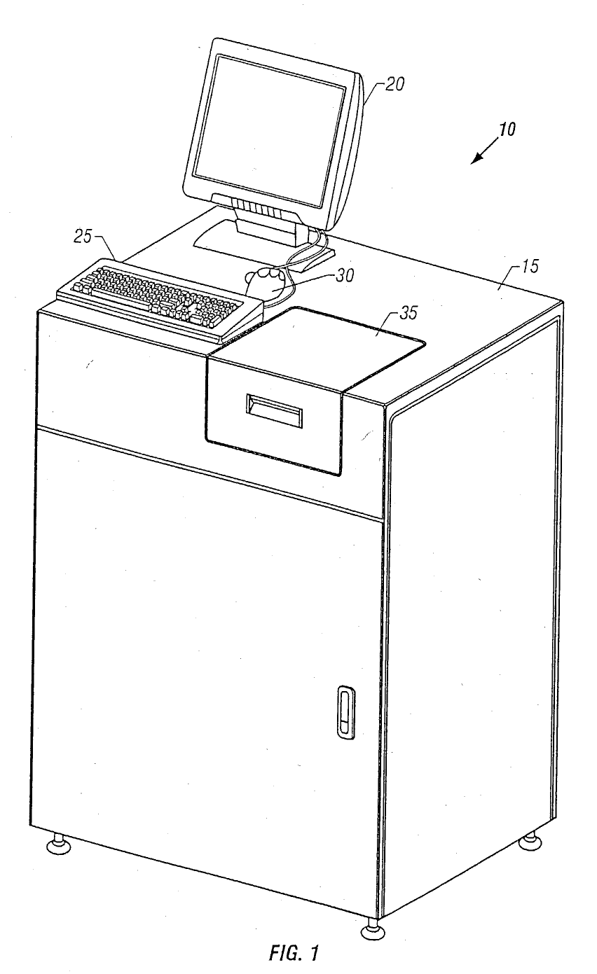

FIG. 1 is an illustration of an exemplary apparatus useful for processing physical entities 10. The physical entity processing apparatus 10 includes a housing 15 that stores the inner components of the apparatus. The housing includes laser safety interlocks to ensure safety of the user, and also limits interference by external influences (e.g., ambient light, dust, etc.). Located on the upper portion of the housing 15 is a display unit 20 for displaying captured images of cell populations in a three-dimensional environment during treatment. These images are captured by a camera array, as will be discussed more specifically below. A keyboard 25 and mouse 30 are used to input data and control the apparatus. An access door 35 provides access to a movable stage that holds a domain containing physical entities undergoing processing.

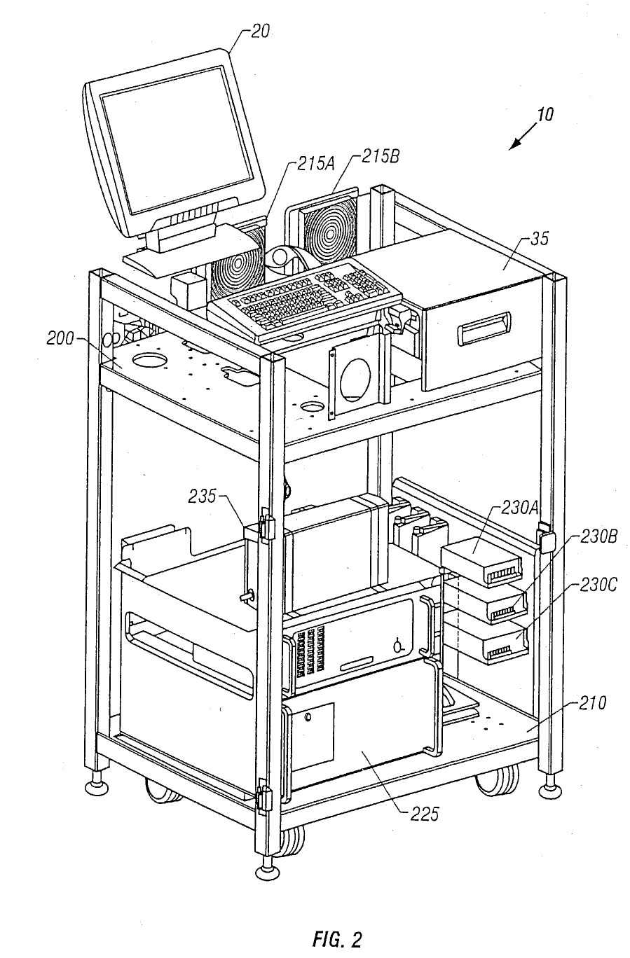

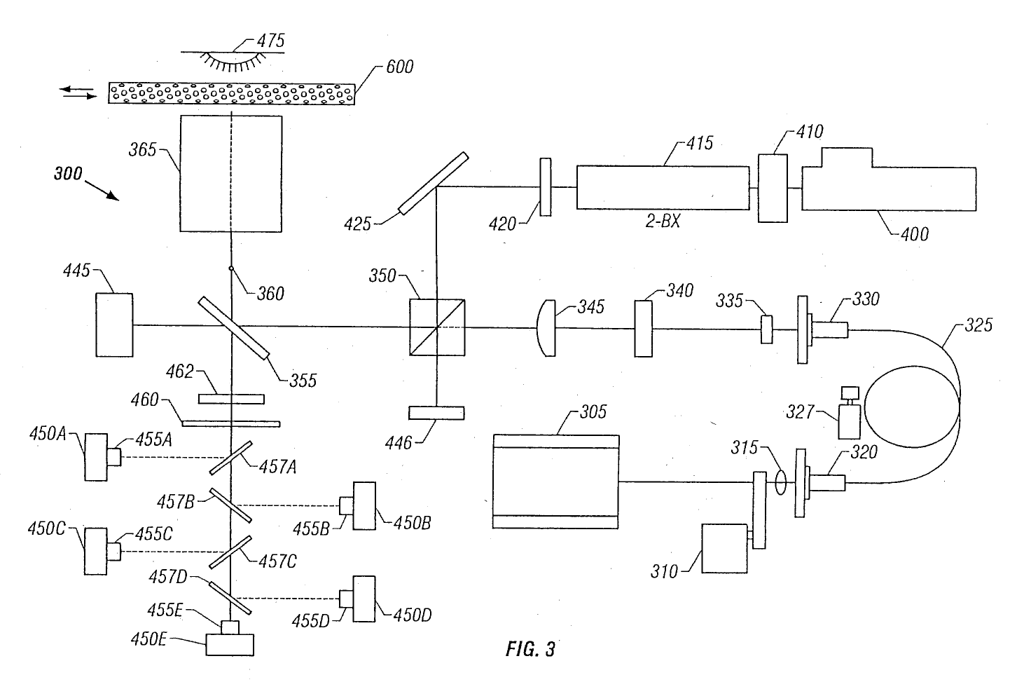

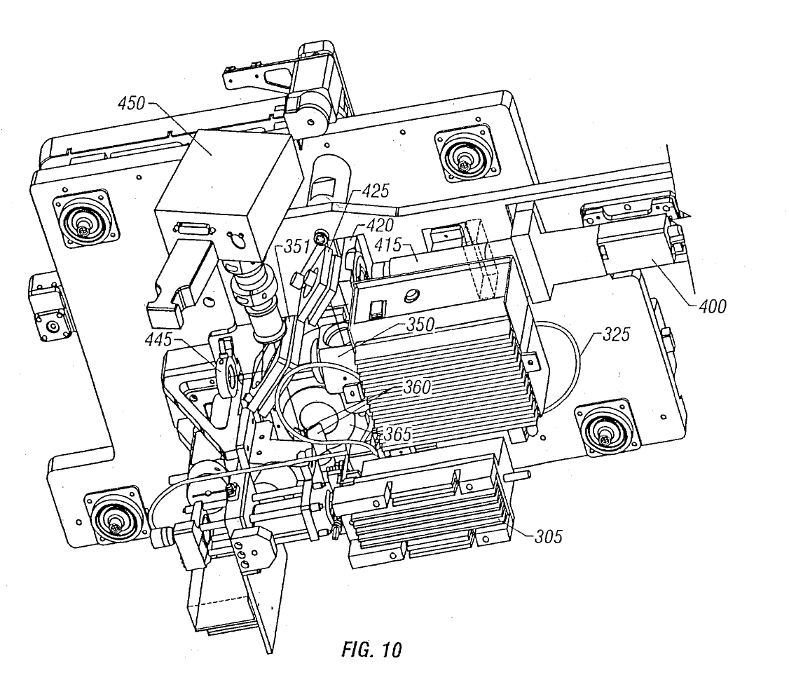

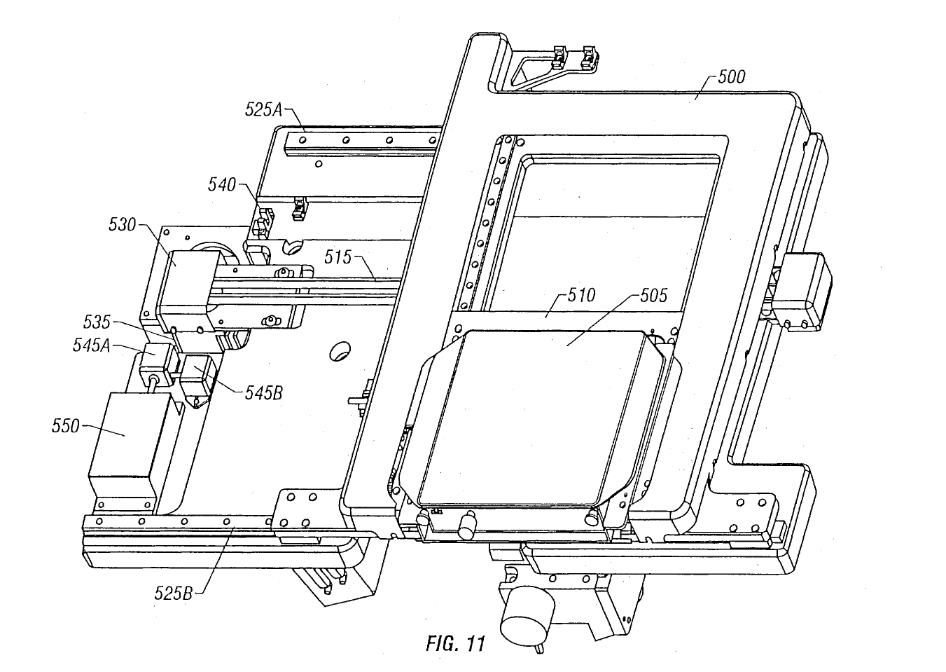

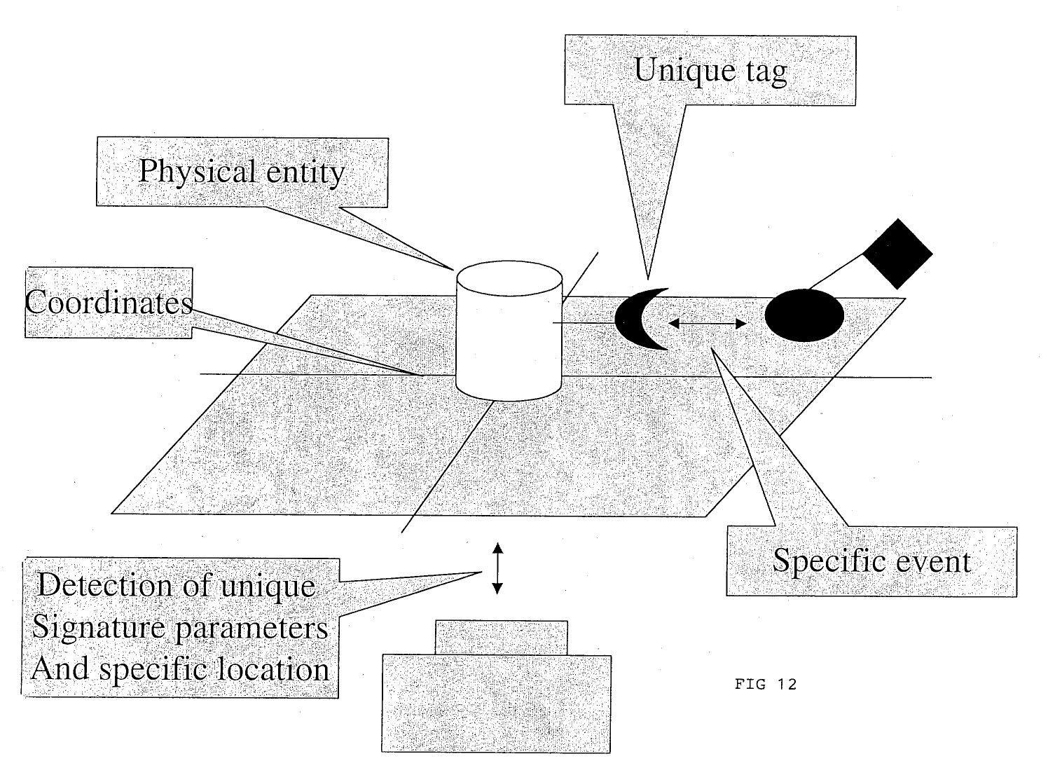

An interior view of the apparatus 10 is provided in FIG. 2. As illustrated, the apparatus 10 provides an upper tray 200 and lower tray 210 that hold the interior components of the apparatus. The upper tray 200 includes a pair of intake filters 215A and B that filter ambient air being drawn into the interior of the apparatus 10. Below the access door 35 is the optical subassembly which is mounted to the upper tray 200 and is discussed in greater detail below with regard to FIGS. 3 through 10.

On the lower tray 210 is a computer 225 which stores the software programs, commands and instructions that run the apparatus 10. In addition, the computer 225 provides control signals to the treatment apparatus through electrical signal connections for steering the laser to the appropriate spot on the domain in order to process the physical entities.

As illustrated, a series of power supplies 230A,B and C provide power to the various electrical components within the apparatus 10. In addition, an uninterruptable power supply 235 can be incorporated to allow the apparatus to continue functioning through short external power interruptions.

FIG. 3 provides a layout of one embodiment of an optical subassembly design 300 for an embodiment of a cell treatment apparatus 10. As illustrated, an illumination laser 305 provides a directed laser output that is used to excite a particular label that is attached to physical entities within a domain. The illumination laser can emit light at various wavelengths in order to optically excite specific labels. Once the illumination laser has generated a light beam, the light passes into a shutter 310 which controls the pulse length of the laser light.

After the illumination laser light passes through the shutter 310, it enters a ball lens 315 where it is focused into an SMA fiber optic connector 320. After the illumination laser beam has entered the fiber optic connector 320, it is transmitted through a fiber optic cable 325 to an outlet 330. By passing the illumination beam through the fiber optic cable 325, the illumination laser 305 can be positioned anywhere within the physical entity processing apparatus and thus is not limited to only being positioned within a direct light pathway to the optical components. The fiber optic cable 325 is connected to a vibrating motor 327 for the purpose of mode scrambling and generating a more uniform illumination spot.

After the light passes through the outlet 330, it is directed into a series of condensing lenses in order to focus the beam to the proper diameter for illuminating one frame of physical entities. As used herein, one frame of physical entities is defined as the portion of the domain that is captured within one image captured by a single camera. This is described more specifically below.

Accordingly, the illumination laser beam passes through a first condenser lens 335. The first lens can have a variety of focal lengths, such as a 4.6 mm focal length. The light beam then passes through a second condenser lens 340 which can have a variety of focal lengths, such as a 100 mm focal length. Finally, the light beam passes into a third condenser lens 345, which provides a 200 mm focal length. Other similar lens configurations that focus the illumination laser beam to an advantageous diameter would function similarly. Thus, this apparatus is not limited to the specific implementation of any particular condenser lens system.

Once the illumination laser beam passes through the third condenser lens 345, it enters a cube beamsplitter 350 that transmits the 532 nm wavelength of light emanating from the illumination laser. Preferably, the cube beamsplitter 350 is a 25.4 mm square cube (Melles-Griot, Irvine, Calif.). However, other sizes are anticipated to function similarly. In addition, a number of plate beamsplitters or pellicle beamsplitters could be used in place of the cube beamsplitter 350 to suit other embodiments. Those skilled in the art will be able to use beamsplitters having a variety of different transmission wavelengths according to the particular labels used, and wavelengths of the illumination laser and transmission laser.

Once the illumination laser light has been transmitted through the cube beamsplitter 350, it reaches a long wave pass mirror 355 that reflects the 532 nm illumination laser light to a set of galvanometer mirrors 360 that steer the illumination laser light, under computer control, through a scanning lens (Special Optics, Wharton, N.J.) 365 to a domain. The galvanometer mirrors are controlled so that the illumination laser light is directed at the proper portion of the three-dimensional physical entity population in the frame of physical entities to be imaged. The scanning lens can include a refractive lens. It should be noted that the term “scanning lens” as used herein contains, but is not limited to, a system of one or more refractive or reflective optical elements used alone or in combination. Further, the scanning lens may include a system of one or more diffractive elements used in combination with one or more refractive and/or reflective optical elements. One skilled in the art will know how to design a scanning lens system in order to illuminate the proper physical entity population.

The light from the illumination laser is of a wavelength that is useful for illuminating the domain. Energy from a continuous wave 532 nm Nd:YAG frequency-doubled laser (B&W Tek, Newark, Del.) reflects off the long wave pass mirror (Custom Scientific, Phoenix, Ariz.) 355 and excites fluorescent labels in the domain. A variety of fluorescent tags can be used. Exemplary fluorescent tags phycoerythrin and Alexa 532 have emission spectra with peaks near 580 nm, so that the emitted fluorescent light from the domain is transmitted via the long wave pass mirror into the camera array. The use of the filter in front of the camera array blocks light that is not within the wavelength range of interest, thereby reducing the amount of background light entering the camera array. Those skilled in the art will be able to select appropriate filters based on the excitation wavelength, excitation and emission spectra of the label used and the optical properties of the long pass filter 355.

The 532 nm illumination laser is further capable of exciting multiple fluorochromes that will emit energy at different wavelengths. For example, PE, Texas Red@, and CyChrome™ can all be efficiently excited by a 532 nm laser. However, they emit energy with spectra that peak at 576 nm, 620 nm, and 670 nm, respectively. This difference in transmitted wavelengths allows the signal from each fluorochrome to be distinguished from the others. In this case, the range of wavelengths transmitted by the filter 460 is expanded. In addition, the camera array is used to capture the emitted light, so that the different signals are distinguished by the computer. Alternatively, the emitted light can be directed to three monochromatic cameras, each having a filter for selective observation of one of the specific fluorochrome's emission wavelengths. Fluorochromes having a variety of differing excitation and emission spectra can be used with appropriate filters and illumination sources to allow detection and differentiation of multiple signals from a single domain. Those skilled in the art will be able to select fluorochromes that can be differentiated by a particular set of optical components by comparison of the excitation and emission spectra for the fluorochromes with consideration for the known illumination and detection wavelengths for the optical components.

A single fixed filter 460 can be replaced with a movable filter cassette or wheel that provides different filters that are moved in and out of the optical pathway. In this way, fluorescent images of different wavelengths of light are captured at different times during physical entity processing. The images are then analyzed and correlated by the computer, providing multicolor information about each physical entity or the population of physical entities as a whole.

It is generally known that many other devices can be used in this manner to illuminate a domain, including, but not limited to, a lamp such as an arc lamp or quartz halogen lamp. Examples of arc lamps useful in the invention include mercury arc lamps or xenon arc lamps. One skilled in the art will know that an appropriate lamp can be chosen based on a variety of factors including average radiance across the spectrum, radiance in specific regions of the spectrum, presence of spectral lines, radiance at spectral lines, or arc size. A light-emitting diode (LED) or laser other than the Nd:YAG frequency-doubled laser described above can also be used in the invention. Thus, the apparatus can use an ion laser such as argon ion or krypton ion laser, Helium neon laser, Helium cadmium laser, dye laser such as a rhodamine 6G laser, YAG laser or diode laser. One skilled in the art can choose an appropriate laser or lamp according to desired properties such as those described above or in Shapiro, Practical flow cytometry, 3^{rd} Ed. Wiley-Liss, New York (1995).

Advantages of the Nd:YAG frequency-doubled laser described above include high intensity, relatively efficient use of energy, compact size, and low generation of heat. It is also generally known that other fluorochromes with different excitation and emission spectra could be used in such an apparatus with the appropriate selection of illumination source, filters, and long and/or short wave pass mirrors. For example, Red®, allophycocyanin (APC), and PharRed™ could all be excited with a 633 nm HeNe illumination laser, whereas fluoroisothiocyanate (FITC), PE, and CyChrome™ could all be excited with a 488 nm Argon illumination laser. One skilled in the art could utilize many other optical layouts with various components in the invention in order to illuminate physical entities so that they return fluorescent energy in multiple wavelengths. The illumination sources described above can be used alone or in combination with other sources to provide a wide variety of illumination wavelengths within a single instrument, thereby allowing the use of many distinguishable labels simultaneously.

The aparatus can be configured to illuminate the domain in any wavelength or wavelength range between 100 nanometers and 30 micrometers including ultra violet (UV) which occurs in the range of about 200 to 390 nm, visible (VIS) occurring in the range of about 390 to 770 nm, and infrared (IR) in the range of about 0.77 to 25 micrometers. A particular wavelength or wavelength range can be produced from a radiation source having a specified output range as described above. As also exemplified above, appropriate optical filters can be chosen to selectively pass, reflect or block radiation based on wavelength. Optical filters useful in the invention include interference filters in which multiple layers of dielectric materials pass or reflect radiation according to constructive or destructive interference between reflections from the various layers. Interference filters are also referred to in the art as dichroic filters, or dielectric filters. Also useful are absorptive filters which prevent passage of radiation having a selective wavelength or wavelength range by absorption. Absorptive filters include colored glass or liquid.

A filter used in the apparatus can have one or more particular filter transmission characteristics including, bandpass, short pass and long pass. A band pass filter selectively passes radiation in a wavelength range defined by a center wavelength of maximum radiation transmission (Tₘₐₓ) and a bandwidth and blocks passage of radiation outside of this range. Tₘₐₓ defines the percentage of radiation transmitted at the center wavelength. The bandwidth is typically described as the full width at half maximum (FWHM) which is the range of wavelengths passed by the filter at a transmission value that is half of Tₘₐₓ. A band pass filter useful in the invention can have a FWHM of 10 nanometers (nm), 20 nm, 30 nm, 40 nm or 50 nm. A long pass filter selectively passes higher wavelength radiation as defined by a Tₘₐₓ and a cut on wavelength. The cut on wavelength is the wavelength at which radiation transmission is half of Tₘₐₓ, and as wavelength increases above the cut on wavelength transmission percentage increases and as wavelength decreases below the cut on wavelength transmission percentage decreases. A short pass filter selectively passes lower wavelength radiation as defined by a Tₘₐₓ and a cut off wavelength. The cut off wavelength is the wavelength at which radiation transmission is half of Tₘₐₓ, and as wavelength increases above the cut off wavelength transmission percentage decreases and as wavelength decreases below the cut off wavelength transmission percentage increases. A filter of the invention can have a Tₘₐₓ of 50-100%, 60-90% or 70-80%.

In addition to the illumination laser 305, a targeting laser 400 is present to irradiate the targeted physical entities once they have been identified by the detector. The radiation beam from the targeting laser can induce an alteration in physical entities within the population of physical entities. As shown, the targeting laser 400 can output an energy beam that passes through a shutter 410. A variety of electromagnetic radiation sources, such as those described above with respect to the illumination source, can also be used and can be selected according to the particular alteration desired in the targeted physical entity.

Once the targeting laser energy beam passes through the shutter 410, it enters a beam expander (Special Optics, Wharton, N.J.) 415 which adjusts the diameter of the energy beam to an appropriate size at the plane of the domain. Following the beam expander 415 is a half-wave plate 420 which controls the polarization of the beam. The targeting laser energy beam is then reflected off a fold mirror 425 and enters the cube beamsplitter 350. The targeting laser energy beam is reflected by 90° in the cube beamsplitter 350, such that it is aligned with the exit pathway of the illumination laser light beam. Thus, the targeting laser energy beam and the illumination laser light beam both exit the cube beamsplitter 350 along the same light path. From the cube beamsplitter 350, the targeting laser beam reflects off the long wave pass mirror 355, is steered by the galvanometers 360, thereafter enters the scanning lens 365 which focuses the targeting electromagnetic radiation beam to a focal volume within the three-dimensional domain. The focal volume receives a sufficient amount of electromagnetic radiation energy to alter a physical entity within the focal volume. However, physical entities in the envelope surrounding the focal volume are not substantially affected by the radiation from the treatment laser.

Thus, physical entities in the envelope surrounding the focal volume are not altered by the targeting laser. However, a focal volume need not entirely encompass a physical entity of interest such that a physical entity of interest having at least a portion within the focal volume can be substantially electromagnetically affected or altered.

It should be noted that a small fraction of the illumination laser light beam passes through the long wave pass mirror 355 and enters a power meter sensor (Gentec, Palo Alto, Calif.) 445. The fraction of the beam entering the power sensor 445 is used to calculate the level of power emanating from the illumination laser 305. In an analogous fashion, a small fraction of the treatment laser energy beam passes through the cube beamsplitter 350 and enters a second power meter sensor (Gentec, Palo Alto, Calif.) 446. The fraction of the beam entering the power sensor 446 is used to calculate the level of power emanating from the treatment laser 400. The power meter sensors are electrically linked to the computer system so that instructions/commands within the computer system capture the power measurement and determine the amount of energy that was emitted from the treatment laser. Thus, the system provides feedback control for altering the power of each laser to suit a particular application.

The energy beam from the targeting laser is of a wavelength that is useful for achieving an alteration of a physical entity. For example, the radiation source can produce a focal volume having sufficient energy to destroy a physical entity. More specifically, a pulsed 523 nm Nd:YLF frequency-doubled laser can be used to heat a localized volume of fluid containing the targeted physical entity, such that it is destroyed. The rate and efficiency of physical entity destruction is dependent upon the actual temperature achieved in the physical entity.

A Nd:YLF frequency-doubled, solid-state laser (Spectra-Physics, Mountain View, Calif.) is used because of its stability, high repetition rate of firing, and long time of maintenance-free service. An energy absorbing dye can be used to reduced the amount of energy required for destroying a physical entity since more of the targeting laser energy is absorbed in the presence of such a dye. One skilled in the art can identify other laser/dye combinations that would result in efficient absorption of energy by the physical entity. For example, a 633 nm HeNe laser's energy would be efficiently absorbed by FD&C green #3 (fast green FCF). Alternatively, a 488 nm Argon laser's energy would be efficiently absorbed by FD&C yellow #5 (sunset yellow FCF), and a 1064 run Nd:YAG laser's energy would be efficiently absorbed by Filtron (Gentex, Zeeland, Mich.) infrared absorbing dye.

An apparatus of the invention can include one or more targeting electromagnetic radiation beams as described above. For example, a plurality of electromagnetic radiation beams can originate from one or more electromagnetic radiation source such as a lamp or laser that is divided and redirected in a plurality of paths. The paths can end in a single focal volume or a plurality of focal volumes. Each path can pass through different optical components to produce treatment electromagnetic radiation beams of differing wavelength or intensity if desired. Alternatively or additionally, a plurality of treatment lasers can be used in an apparatus of the invention.

More than one laser can be directed to a domain such that the electromagnetic radiation beams intersect at a focal volume within the domain. The focal volume at which the electromagnetic radiation beams intersect will experience a higher intensity of radiation than other regions within the envelope surrounding the focal volume. The intensity and number of electromagnetic radiation beams intersecting the domain can be selected to produce sufficient combined energy to electromagnetically affect a particle within the focal volume while individually producing an amount of energy that is not capable of substantially electromagnetically affecting particles outside of the focal volume. Two or more targeting electromagnetic radiation beams that intersect a focal volume of a domain or physical entity can have differing wavelengths and can irradiate a focal volume simultaneously or sequentially as desired to induce a particular electromagnetic effect or combination of electromagnetic effects. The wavelengths and intensities of the electromagnetic radiation beams can be selected from within the ranges described previously.

In addition to the illumination laser 305 and targeting laser 400, the apparatus includes a detector having an array of cameras 450A, 450B, 450C and 450D that capture images, or frames of the cell populations at stepped Z-levels (Z-levels are also referred to herein as depths of field). The camera array contains a plurality of cameras having views offset vertically with respect to each other which allows the array to capture physical entity images at various Z-levels, or depths, within the domain. As illustrated in FIG. 3, each camera 450A, 450B, 450C, 450D and 450E is focused through a lens 455A, 455B, 455C, 455D and 455E, respectively to capture light reflected by a beamsplitter 457A, 457B, 457C and 457D, respectively. Prior to reaching the beamsplitters 457A, 457B, 457C and 457D the light from the domain passes through a filter 460 to allow accurate imaging of the physical entities at the desired wavelengths without capturing stray background light occurring at other wavelengths. A stop 462 is positioned between the filter 460 and mirror 355 in order to prevent unwanted light from entering the camera array from angles not associated with the image from the domain. The filter 460 is chosen to selectively pass light within a certain wavelength range. The wavelength range of transmitted light includes wavelengths emitted from the targeted physical entities upon excitation by the illumination laser 305, as well as those from a back-light source 475. The filter 460 selectively prevents passage of light in the wavelength region of the illumination laser which would otherwise saturate the detector or render the fluorescence signal undetectable.

The back-light source 475 is located above the domain 600 to provide back-illumination of the domain at a wavelength different from that provided by the illumination laser 305. In the example described here, the back light source is an LED that emits light at 590 nm, such that it can be transmitted through the long wave pass mirror to be directed into the camera array. This back-illumination is useful for imaging physical entities whether or not there are fluorescent targets within the frame being imaged. The back-light can be used in attaining proper focus of the system, even when the physical entities do not contain fluorescent labels. In one example set-up, the back-light is mounted on the underside of the access door 35 (FIG. 2). Thus, the apparatus can be configured with an appropriate back light, illumination laser and optical filters to selectively pass illumination of a desired wavelength to the camera array. Other wavelengths of light are prevented from passing through the filter 460, and being recorded by the camera array 450.

It should be noted that in the presently described apparatus, the detector includes a camera array having a plurality of charge-coupled devices (CCD). The cameras can be placed to view different focal planar regions, each of the viewed focal planar regions being a different sectional image of the domain. The detector can transmit the sectional images back to the computer system for processing. As will be described below, the computer system determines the coordinates of the targeted physical entities in the domain by reference to one or more sectional images captured by the CCD camera array.

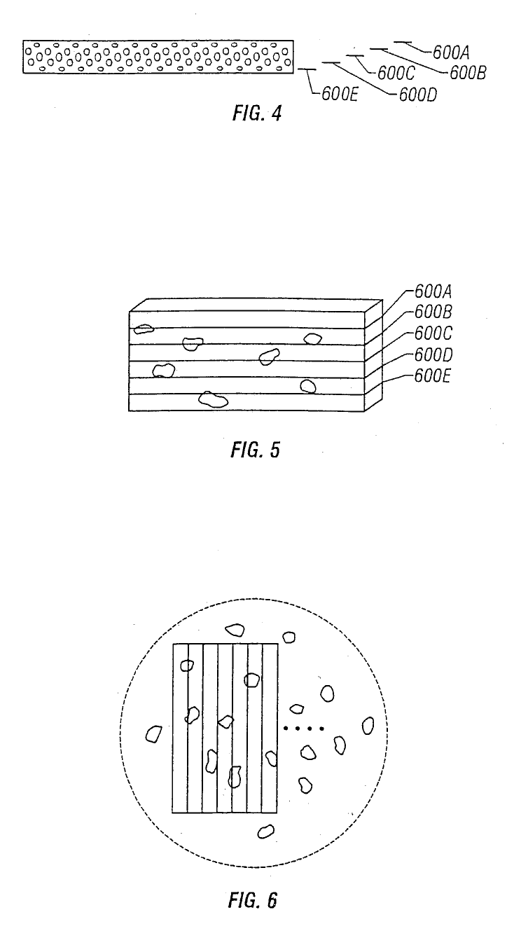

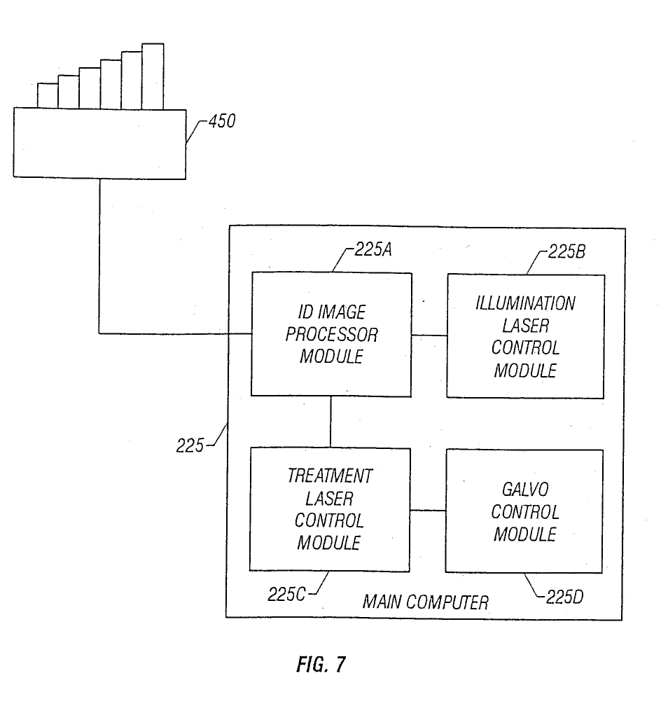

Referring generally to FIGS. 4 through 6, the use of the CCD camera array is illustrated. As illustrated, the views of the CCD cameras are substantially parallel and each CCD camera views a different focal length. Different focal planes can be viewed by the cameras by vertically offsetting each camera within the array or by placing focusing optics between the camera and domain. Using such an arrangement it becomes possible to capture focused images of physical entities within focal planar regions observed at different depths of field within the domain. As illustrated in FIG. 5, the focal planar regions observed at each depth of field, as indicated by sections 600A through 600E, can be captured as sectional images and then assembled by a three-dimensional image processor 225A of FIG. 7 to produce a three-dimensional volume image of the domain. This image is then used to determine three coordinates for aiming the targeting laser to the appropriate location within the volume of the domain.

The apparatus can produce sectional images at a variety of depths of field according to the configuration of optical devices. Those skilled in the art will be able to configure the detector to image at a shallow depth of field which includes a depth of less than 100 microns. Depending upon the size of the domain the depth of field can be selected from 1, 2, 3, 4, 5, 6, 7, 8, 9, 10, 11, 12, 15, 20, 25, 30, 35, 40, 45, 50, 60, 70, 80 90 or 100 microns. For larger domains even greater depths of field can be employed for deeper imaging.

The apparatus of the invention can be configured to capture images of the domain at different resolutions or magnifications. This can be achieved by altering the property of the lens 455 in front of one or more cameras. A turret, cassette or wheel containing different lenses can be placed between the camera and domain such that the magnification or resolution can be rapidly changed. The turret, cassette or wheel can be functionally attached to a positioning device for manual or automated changes in resolution or magnification during the course of or between domain processing procedures.

A detector used in the apparatus can also include two or more cameras capable of imaging the domain from different directions of view. Imaging from different directions of view, also referred to as stereo-imaging, can be used to reconstruct an image of the domain. Two or more cameras can stereo-image a domain when their different directions of view are separated by an angle selected from 1 to 180 degrees. The apparatus can include cameras having different directions of view separated by less than 1 degree, 2 degrees, 5 degrees, 10 degrees, 15 degrees, 20 degrees, 25 degrees, 30 degrees, 35 degrees, 40 degrees, 45 degrees, 50 degrees, 90 degrees or 180 degrees, wherein a degree is intended to be used consistent with mathematical usage wherein it is an angle subtending 1/360 of the circumference of a circle.

A detector used in the apparatus can include one or more cameras viewing a relatively shallow focal planar region, wherein the focal planar region can be refocused on different sections of the domain. A particular camera view can be refocused to observe a domain at different depths of field thereby obtaining different sectional images of the domain. Such refocusing can be achieved by moving the camera. Other lower inertia components of the detector are preferably moved in order to achieve refocus and include lenses or mirrors placed in between the optical path of the camera and domain. The component to be adjusted can be operably attached to a positioning device for manual or automated refocusing. Automated focusing can be achieved by incorporation of an automated positioning device that is capable of communicating with imaging processing devices such as those described below.

Any detector capable of converting radiation directed from a domain or particle therein into a signal that can be subsequently manipulated or stored to determine the presence or quantity of a particle in a domain can be used in the apparatus or methods of the invention. A detector can include a photodiode, photomultiplier tube or charge-coupled device. A detector can also include an imaging device that converts radiation directed from a domain or particle therein to a set of signals that can be converted into a 3-dimensional representation of a domain. Such an imaging device can include a camera such as a CCD camera, digital camera, film camera or photographic camera and the like. One skilled in the art will be able to choose a detector based on a variety of well known factors including, for example, compatibility with the radiation source used, sensitivity, spectral range of detection and compatibility with data processing devices.

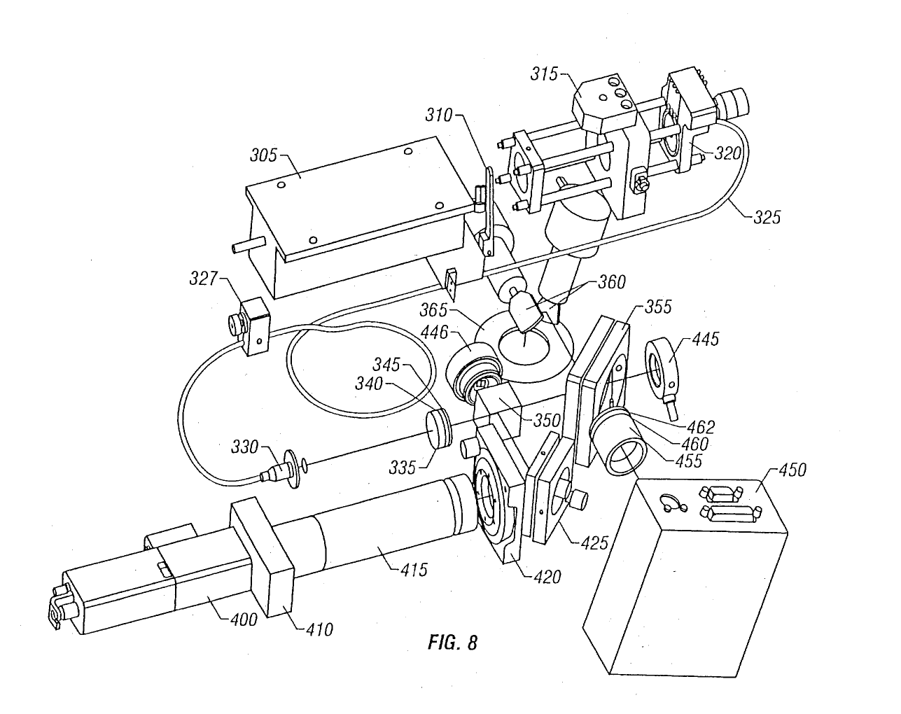

Referring now to FIG. 8, a perspective view of an example of an optical subassembly is illustrated. As illustrated, the illumination laser 305 sends a light beam through the shutter 310 and ball lens 315 to the SMA fiber optic connector 320. The light passes through the fiber optic cable 325 and through the output 330 into the condenser lenses 335, 340 and 345. The light then enters the cube beamsplitter 350 and is transmitted to the long wave pass mirror 355. From the long wave pass mirror 355, the light beam enters the computer-controlled galvanometers 360 and is then steered to the proper frame of cells in the domain through the scanning lens 365.

As also illustrated in the perspective drawing of FIG. 8, the targeting laser 400 transmits energy through the shutter 410 and into the beam expander 415. Energy from the targeting laser 400 passes through the beam expander 415 and passes through the half-wave plate 420 before hitting the fold mirror 425 and subsequently entering the cube beamsplitter 350 where it is reflected 90° to the long wave pass mirror 355, from which it is reflected into the computer controlled galvanometer mirrors 360. The galvanometer mirrors 360 can be adjusted to steer the targeting laser beam through the scanning lens 365 such that the beam strikes the portion of a domain where a particular target physical entity is located. Accordingly, a desired response can be selectively induced in the target physical entity using the apparatus.

In order to accommodate a very large surface area of domain, the apparatus includes a movable stage that mechanically moves the domain with respect to the scanning lens. Thus, once a specific sub-population of physical entities within the scanning lens field-of-view has been treated, the movable stage brings another sub-population of physical entities within the scanning lens field-of-view. As illustrated in FIG. 11, a computer-controlled movable stage 500 holds a domain container 505 which contains a domain 600 to be processed. The movable stage 500 is moved by computer-controlled servo motors along two axes so that the domain can be moved relative to the optical components of the instrument. The stage movement along a defined path is coordinated with other operations of the apparatus. In addition, specific coordinates can be saved and recalled to allow, return of the movable stage to positions of interest. Encoders on the x and y movement provide closed-loop feedback control of stage position.

A flat-field (F-theta) scanning lens 365 can be mounted below the movable stage. The scanning lens field-of-view comprises the portion of the domain that is presently positioned above the scanning lens by the movable stage 500. The lens 365 can be mounted to a stepper motor that allows the lens 365 to be automatically raised and lowered (along the z-axis) for the purpose of focusing the system.

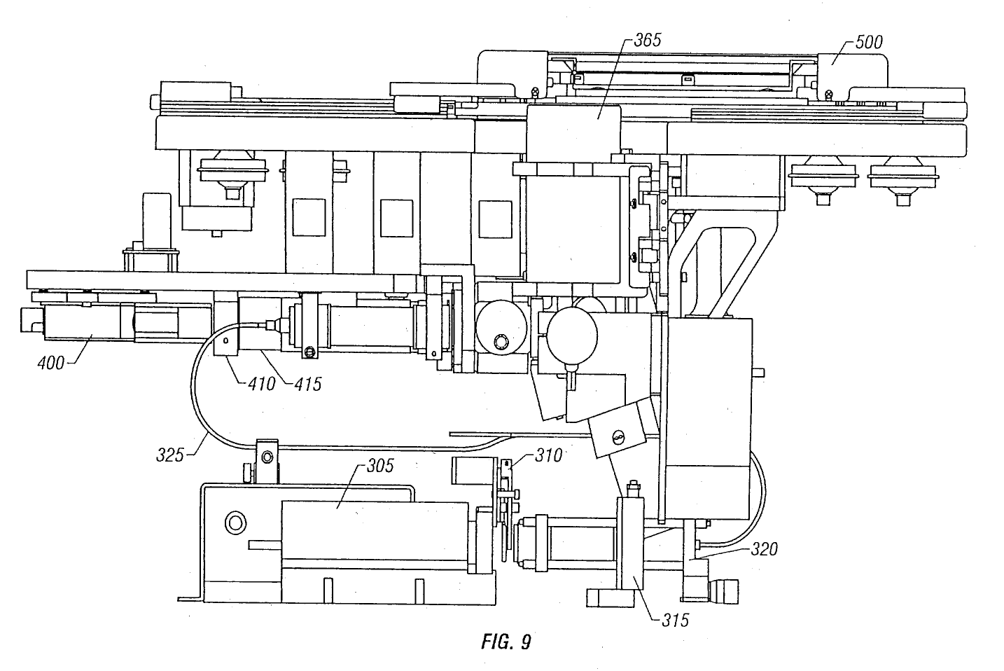

As illustrated in FIGS. 8-10, below the scanning lens 365 are the galvanometer-controlled steering mirrors 360 that deflect electromagnetic energy along two perpendicular axes. Behind the steering mirrors is the long wave pass mirror 355 that reflects electromagnetic energy of a wavelength shorter than 545 nm. Wavelengths longer than 545 nm are passed through the long wave pass mirror, directed through the filter 460, coupling lens 455, and into the CCD camera array, thereby producing an image of the appropriate size on the CCD sensor of the camera array 450 (See FIGS. 3 and 4). The magnification defined by the combination of the scanning lens 365 and coupling lens 455 can be chosen to reliably detect single cells while maximizing the area viewed in one frame by each camera. Although a CCD camera array (DVC, Austin, Tex.) is illustrated in this example, the camera can be any type of detector or image gathering equipment known to those skilled in the art, as described above. The optical subassembly of the apparatus is preferably mounted on a vibration-damping platform to provide stability during operation as illustrated in FIGS. 2 and 9.

Referring now to FIG. 11, a top view of the movable stage 500 is illustrated. As shown, a domain can be detachably mounted in the movable stage 500. The domain 505 rests on an upper axis nest plate 510 that is designed to move in the forward and backward direction with respect to the movable stage 500. A stepper motor can be connected to the upper axis nest plate 510 and computer system so that commands from the computer direct forward or backward movement of the domain container 505.

The movable stage 500 is also connected to a timing belt 515 that provides side-to-side movement of the movable stage 500 along a pair of bearing tracks 525A and B. The timing belt 515 attaches to a pulley housed under a pulley cover 530. The pulley is connected to a stepper motor 535 that drives the timing belt 515 to result in side-to-side movement of the movable stage 500. The stepper motor 535 is electrically connected to the computer system so that commands within the computer system control side-to-side movement of the movable stage 500. A travel limit sensor 540 connects to the computer system and causes an alert if the movable stage travels beyond a predetermined lateral distance.

A pair of accelerometers 545A and B is preferably incorporated on this platform to register any excessive bumps or vibrations that may interfere with the apparatus operation. In addition, a two-axis inclinometer 550 is preferably incorporated on the movable stage to ensure that the domain container is level, thereby reducing the possibility of gravity-induced motion in the domain container.

The domain chamber has a fan with ductwork to eliminate condensation on the domain container, and a thermocouple to determine whether the domain chamber is within an acceptable temperature range. Additional fans are provided to expel the heat generated by the electronic components, and appropriate filters are used on the air intakes 215A and B (see FIG. 2).

The computer system 225 controls the operation and synchronization of the various components of electronic hardware described above. The computer system can be any commercially available computer that can interface with the hardware. One example of such a computer system is an Intel Pentium® IV-based computer running the Microsoft Windows® 2000 operating system. Software is used to communicate with the various devices, and control the operation in the manner that is described below.

Once a domain is in place on the movable stage and the door is closed, the computer passes a signal to the stage to move into a home position. The fan is initialized to begin warming and defogging of the domain. During this time, physical entities within the domain are allowed to settle to the bottom surface. In addition, during this time, the apparatus may run commands that ensure that the domain is properly loaded, and is within the focal range of the system optics. For example, specific markings on the domain container can be located and focused on by the system to ensure that the scanning lens has been properly focused on the bottom of the domain container. After a suitable time, the computer turns off the fan to prevent excess vibrations during treatment, and physical entity processing begins.

First, the computer instructs the movable stage to be positioned over the scanning lens so that the first area of the domain to be treated is directly in the scanning lens field-of-view. The galvanometer mirrors are instructed to move such that the center frame within the field-of-view is imaged in the camera. As discussed below, the field imaged by the scanning lens is separated into a plurality of frames. Each frame is the proper size so that the physical entities within the frame are effectively imaged by the camera array.

The back-light 475 is then activated in order to illuminate the field-of-view so that it can be brought into focus by the scanning lens. Once the scanning lens has been properly focused upon the domain, the computer system divides the field-of-view into a plurality of frames so that each frame is analyzed separately by the camera array. This methodology allows the apparatus to process a plurality of frames within a large field-of-view without moving the mechanical stage. Because the galvanometers can move from one frame to the next very rapidly compared to the mechanical steps involved in moving the stage, this method results in an extremely fast and efficient apparatus.

The apparatus can further include an image processing device 225A for combining one or more two-dimensional representations of a domain and producing a three-dimensional representation. A two-dimensional or three-dimensional representation refers to an image or any characterization of a domain, or portion thereof, that specifies the coordinates of at least one physical entity of interest therein such as a graphical or tabular list of coordinates or a set of computer commands that can be used to produce an image.

Initially, one or more two-dimensional representations such as two-dimensional sectional images can be captured by the camera array and stored to a memory in the computer. Although, a single two-dimensional image can contains sufficient information to produce a three dimensional representation of a domain, it may be desirable to process two or more or a plurality of two-dimensional images to produce a three-dimensional image. Instructions in the computer can produce or calculate a three dimensional representation such as a three-dimensional image. A three-dimensional image calculated as such can be analyzed with respect to the size, shape, number, or other object features in the image at each stepped Z-level. If necessary, the computer instructs the z-axis motor attached to the scanning lens to raise or lower in order to improve focus on the frame of interest. The galvanometer-controlled mirrors are then instructed to image a first frame, within the field-of-view, in the camera array. Once the galvanometer mirrors are pointed to the first frame in the field-of-view, the shutter in front of the illumination laser is opened to illuminate the first frame through the galvanometer mirrors and scanning lens. The camera array captures an image of any fluorescent emission from the domain in the first frame of cells. Once the image has been acquired, the shutter in front of the illumination laser is closed and a software program (Epic, Buffalo Grove, Ill.) within the computer processes the image.

The image processing device 225A can include the capability of virtual autofocusing by searching sectional images of a domain and identifying a sectional image that is in-focus. Virtual autofocusing does not require production of a three-dimensional representation of any part of the domain and can, therefore, be performed prior to or absent formation of a three-dimensional representation. A plurality of sectional representations such as sectional images can be obtained as described above using one or more cameras viewing different focal planar regions. Virtual autofocusing can be achieved by analyzing multiple sectional images and selecting an in-focus image. Subsequent image processing can then be selectively carried out for the in-focus sectional image in order to efficiently identify a desired target particle. Thus, a particle of interest can be identified or located in a domain based on its X and Y coordinates in the in-focus sectional image and the Z-level of the sectional image. The X and Y coordinates as used herein refer to coordinates in two dimensions forming a plane orthogonal to the direction of view. A plurality of in-focus sectional images selected by virtual autofocusing can be used to calculate a three-dimensional image as described above.

Although real-time autofocusing can be used, virtual autofocusing provides the advantage of more rapid throughput. Specifically, real-time autofocusing often requires multiple adjustments of optical components and re-imaging until an in-focus sectional image is obtained. In contrast, when a plurality of fixed cameras are placed to view non-overlapping focal planar regions, at least one camera will have a focused view without the need to move any component of the detector. Subsequently, the images can be analyzed using algorithms similar to those used in real-time autofocusing methods without the requirement for time-consuming movement of optical components and reacquisition of images.

Known autofocusing algorithms such as those used in microscopy or autofocus cameras can be used to analyze sectional images and identify an in-focus sectional image in the apparatus and methods of the invention. An example of an autofocus method that can be used in the apparatus or methods of the invention is binary search autofocus. Binary search autofocus can be performed virtually by preselecting two sectional images between which an in-focus sectional image is thought to exist and iteratively reducing the number of intervening sectional images until one having a desired focus is identified. The iterations include the, steps of selecting a sectional image that is halfway between the boundary sectional images, evaluating the selected sectional image for a predetermined focus value and further reducing the boundary distance until a sectional image having the desired focus value is identified. Alternatively, a sequential autofocus method can be used in which sectional images are analyzed in a stepwise fashion starting from a preselected initial sectional image.

The detector also can capture an image of a two-dimensional domain. Virtual autofocusing will work if the depth of the domain is less than the depth of field of the detector view. Virtual autofocusing can be carried out as described above to identify or locate the X and Y coordinates for a particle of interest located in the two-dimensional domain. The particle identified as such can be targeted and electromagnetically affected using an apparatus or method of the invention.

The power sensor 445, discussed above, detects the level of light intensity emitted by the illumination laser. Based on the measured intensity, the computer can determine if an appropriate amount of light has illuminated the frame of cells for the particular application. In the event a particular threshold has not been obtained or the signal surpasses a desired maximum the laser intensity can be adjusted and another illumination and image capture sequence performed. Such iteration can be carried out until the appropriate conditions are achieved or after a preselected number of iterations the system can pause or indicate in an error condition that is communicated to the operator.

The threshold or maximum energy levels will depend upon the particular application of the apparatus or methods of the invention. The term “threshold” refers to the amount of energy sufficient to change a particular property. For example, a threshold amount of electromagnetic energy can be an amount sufficient to attach a physical entity to a domain, alter a physical entity density, to increase or decrease pH within a defined range, or to induce a chemical reaction. Other physical entities in the same domain that do not receive radiation at or beyond the threshold will not undergo the particular change. A range of electromagnetic energy used in the apparatus or methods of the invention can be defined by a threshold and a ceiling. A “ceiling” is intended to mean an amount of energy that is greater than a threshold amount and sufficient to induce an unwanted change in a particular property. The ceiling can be defined by any detectable change including those described above in relation to a threshold energy. Thus, a range of electromagnetic energy used in the methods of the invention can include an amount of energy sufficient to induce a chemical reaction on or within a physical entity without causing destruction of the physical entity.

Shuttering of illumination light can be used to reduce undesirable heating and photobleaching of the domain and to provide a fluorescent signal in a desired range of detection. An image analysis algorithm is run to locate the x-y-z centroid coordinates of all targeted physical entities in the frame by reference to features in the captured image. If there are targets in the image, the computer calculates the three-dimensional coordinates of the target locations in relation to the movable stage position and field-of-view, and then positions the galvanometer-controlled mirrors to point the treatment electromagnetic radiation beam to the location of the first target in the first frame of physical entities. It should be noted that the z-coordinate may be calculated by the algorithm based in part upon the focal length of the camera that captured the image. It should further be noted that only a single frame of physical entities within the field-of-view has been captured and analyzed at this point. Thus, there should be a relatively small number of identified targets within this sub-population of the domain. Moreover, because the camera array is pointed to a smaller population of physical entities, a higher magnification is used so that each target is imaged by many pixels within the CCD camera.

Once the computer system has positioned the galvanometer controlled mirrors to point to the location of the first targeted physical entity within the first frame of physical entities, the targeting laser is fired for a brief interval so that the first targeted physical entity is given an appropriate dose of energy. The power sensor 446 discussed above detects the level of energy that was emitted by the targeting laser, thereby allowing the computer to calculate if it was within a desired range to induce a response in the targeted physical entity. The power of the targeting laser can be adjusted and the targeting laser fired at the same target again. The iterative targeting, firing, and sensing steps can be repeated until appropriate conditions are achieved or up to a predetermined number of rounds after which the iteration is paused or an error message communicated to the operator. In addition, the targeting laser can be fired once at more than one, a group or all of the target physical entities within a frame, and subsequently the computer can direct the target laser to return to any physical entities that did not receive a sufficient level of energy to induce an alteration.

Once all of the targets have been irradiated with the targeting laser in the first frame of physical entities, the mirrors can be positioned to the second frame of physical entities in the field-of-view, and the processing repeated at the point of frame illumination and camera imaging. This processing can be continued for all frames within the field-of-view above the scanning lens. When all of these frames have been processed, the computer instructs the movable stage to move to the next field-of-view in the domain, and the process repeated from the back-light illumination and auto-focus steps. Frames and fields-of-view can be overlapped to reduce the possibility of inadvertently missing areas of the domain. Once the domain has been fully processed, the operator is signaled to remove the domain, and the apparatus is immediately ready for the next domain. Although the text above describes the analysis of fluorescent images for locating targets, those skilled in the art will understand that the non-fluorescent back-light LED illumination images can be useful for locating target cells based on other properties such as those viewable in a standard microscope based on absorbance, transmittance or refraction of light.

The galvanometer mirrors provide the advantage of controlling the imaging of successive frames and the irradiation of successive targets. One brand of galvanometer is the Cambridge Technology, Inc. model number 6860 (Cambridge, Mass.). This galvanometer can reposition very accurately within a fraction of a millisecond, making the processing of large areas and many targets possible within a reasonable amount of time. In addition, the movable stage can be used to move specified areas of the domain into the scanning lens field-of-view. This combination of movements can be automated providing increased throughput of the apparatus or methods.

It should be understood that other configurations of an apparatus are also possible. For example, a movable stage, similar to a conveyer belt, could be included to continuously move a domain of physical entities through the above-described process. Error signals continuously generated by the galvanometer control boards are monitored by the computer to ensure that the mirrors are in position and stable before an image is captured, or before a target is fired upon, in a closed-loop fashion.

The methods of the invention can involve applying applying energy to one or more physical entities. Energy can be applied from a variety of energy sources, including diffuse and controlled energy sources. A controlled energy source can be, for example, a laser or lamp. A controlled energy source can provide energy in at least 1, 2, 3, 4, 5, 6, 7, 8, 9, or 10 square centimeters of a domain per minute. The methods can involve processing at least 0.25, 0.5, 1, 2, 3 or 4 million physical entities in a domain per minute. The rate at which physical entities are treated with energy can be measured as the number of physical entity containing focal volumes that are altered per minute. At least 1, 2, 3, 4, 5, 6, 8, 10, 15, 20, 30, 60, 100, 300, 500, 1000, 3000, 5000, 10000, 30000, 50000, 100000, 300000, or 500000 separate focal volumes in the domain per minute can be altered. Furthermore, the rate of imaging can be at the rate of at least 1 Hz, 2 Hz, 3 Hz, 4 Hz, 5 Hz, 6 Hz, 8 Hz, 10 Hz, 15 Hz, 30 Hz, 50 Hz, 100 Hz, 150 Hz, 300 Hz, 500 Hz, or 1000 Hz.

Of course, many variations of the above-described methods are possible, including alternative methods for illuminating, imaging, and targeting the physical entities. For example, movement of the domain relative to the scanning lens could be achieved by keeping the domain substantially stationary while the scanning lens is moved. Steering of the illumination beam, images, and energy beam could be achieved through any controllable reflective or diffractive device, including prisms, piezo-electric tilt platforms, or acousto-optic deflectors.

Additionally, an image can be viewed from either below or above the domain. Because an apparatus can be focused through a movable scanning lens, the illumination and energy beams are directed to different focal planes along the z-axis. Thus, portions of the domain that are located at different vertical heights are specifically imaged and processed by an apparatus in a three-dimensional manner. The sequence of the steps could also be altered without changing the process. For example, one might locate and store the coordinates of all targets in the domain, and then return to the targets to irradiate them with energy one or more times over a period of time.

To optimally process the domain, it should be placed on a substantially flat surface so that a large portion of the domain appears within a narrow range of focus. The density of physical entities on this surface can, in principle, be at any value. However, increasing the density of physical entities can minimize the total surface area required to be scanned or detected using the methods of the invention.

To prepare a population of physical entities having one or more incorporated dyes, methods known in the art can be employed. Typically, a copolymerization process involving polymerization of monomers, such as unsaturated aldehyde or acrylate, in the presence of one or more dyes, such as fluorescent dyes. For example, see U.S. Pat. Nos. 4,267,234; 4,267,235; 4,552,812 and 4,677,138.

It is understood that modifications which do not substantially affect the activity of the various embodiments of this invention are also included within the definition of the invention provided herein. Accordingly, the following examples are intended to illustrate but not limit the present invention.

Throughout this application various publications have been referenced within parentheses. The disclosures of these publications in their entireties are hereby incorporated by reference in this application in order to more fully describe the state of the art to which this invention pertains.

### EXAMPLE I

### Producing Populations of Physical Entities Having Defined Characteristics

This example shows a method for obtaining a population of physical entities having defined characteristics.

To produce populations of physical entities, such as beads, that can be distinguished from each other based on size, an appropriate distribution of bead diameters in each population is determined. For example, if it is desired to obtain beads that are 5 microns in diameter, an appropriate distribution of bead diameters can be between 4 and 6 microns. This population of beads can be distinguished from a second population having a distribution of bead diameters of 9 to 11 microns, for example. Thus, the values of the distinguishing parameter may be within a range of values within a population, so long as the range is clearly discernable from that parameter in other bead populations. Clearly this would apply to a range of values of diameters. Exemplary size ranges for beads include 4 to 6 micron, 9 to 11 micron, 14 to 16 micron, 19 to 21 micron and the like.

Any quantifiable parameter can be graded in a similar fashion. For example, fluorescent probes and quantum dots can be embedded in a physical entity such as a bead. Grading of the response can be done in a similar fashion as for size. All beads with a fluorescent response in a particular range, for example 520 to 540 nm, can be identified and the strength of the fluorescent signal graded in to quantitatively different and non-overlapping intensity levels. This process would generate a set of beads of a particular size and particular fluorescence characteristics. Thus depending on the manufacturing process, beads of unique parameter signature can be made in a one-parameter-at-a-time fashion on multiple parameters at the same time. Depending on the quality of the manufacturing process, a separation process may be required to eliminate physical entities that do not fit the stated numerical criterion for the different parameters. The manufacturing process employed in preparing a population of beads having unique parameter signatures will vary depending on the particular parameter signatures used. For example, a process for preparing beads containing dyes is described in U.S. Pat. Nos. 4,267,234; 4,267,235; 4,552,812 and 4,677,138. Processes for preparing beads having particular size ranges are well known in the art. The methods described herein for selecting a population of non-cellular physical entities can be used to obtain a population of physical entities having desired parameter signatures.

### EXAMPLE II

### A Method for Preparing Uniquely Tagged Probes

This example describes a method for preparing a population of uniquely tagged probes.

A mixture of physical entities, each with a different signature parameter, are placed in different physical locations. For example, a population of beads of multiple sizes are placed into wells of a multi-well plate. Each physical location contains a separate tag present in a soluble form. As each location is being scanned and a physical entity of a particular specification is located, then a separate energy source can be used to chemically associate a physical entity with a tag.

As a numerical example this procedure can be performed using beads of ten different sizes placed in ten different locations. In this case, about 10% of the beads of a particular size are found in each one of the ten locations. A different tag is then placed in liquid solution in each of the ten locations. Then chemical conjugation of the tag, such as an oligonucleotide, can be carried out using a laser focused only on the beads of a given size to chemically attach the tag found in that location to beds of a specific size. Thus, in each location only the tagging of the beads of a particular size is carried out. A number of light-sensitive chemistries have been described in the literature that can accomplish this goal.

This procedure can be carried out in all the separate locations. Then all the physical entities from all the locations can be collected into one population. The physical entities can then be redistributed into the separate physical locations. The unique tags can then be placed again in the physical locations. The tagging procedure described before in this example can then be repeated. Then the physical entities can be pooled again. Now a larger fraction of the physical entities with a particular parameter signature have been tagged. This procedure can thus be repeated until a desirable fraction of the physical entities has been appropriately tagged.

Continuing with the specific numerical example above, the pooled population of ten bead sizes from all ten locations will have abut 10% of a specific bead size with a specific tag on it. Once the beads are placed back in the specific locations each with a specific tag, the remaining 90% of the beads of a particular size can now be tagged in that location using the focused laser. The same procedure is then carried out in each location. Once the beads from all the location is pooled then on the average 19% of the beads of a particular size are properly tagged. A third iteration of the process will add 8.1% of tagged beads in each size group for a total of 27.1% of the beads tagged. This procedure can then be repeated to generate specific tagging of whatever percentage of beads of a particular size that one desires. Economics of the process will determine how often this process is repeated.

Thus, this example describes tagging physical entities with unique probes without ever separating the mixed population of physical entities.

### Example III

### Multiplexing Assays

This example describes the use of physical entities having unique tags, or “unique tags” for multiplexed assays. Beads of three different size are prepared in nominal sizes of 1, 3 and 10 microns. These can be polystyrene beads. These beads can be impregnated with a label of a particular color or be left transparent as they are produced. Thus six unique parameter signatures are made, namely (1 micron, transparent), (3 micron, transparent), (10 micron, transparent), (1 micron, color), (3 micron, color), and (10 micron, color). Each bead can then be attached to a probe, such as an oligonucleotide. A mixture of these six beads can then be exposed to a liquid solution of complementary oligonucleotides labeled with fluorescent tags. Thus six unique hybridization events can be detected simultaneously.

If the color intensity of the beads can be quantitatively graded into 3 different intensity levels (in addition to transparent), then 3×4=12 unique parameter signatures can be obtained. If two colors at three intensities can be impregnated into the beads of three different sizes, then we 4×4×3=48 unique parameter combinations can be obtained.

Thus by increasing the number of characteristics or signature parameters that define the specific signature of the physical entities, and by having the ability to quantitatively grade the readout of each parameter, a large number of unique tags can be obtained. In general n read-out levels of m signature parameters give up to nm unique specific signatures. Thus ten parameters and ten read-out levels would give 10 billion unique specific signatures.

### EXAMPLE IV

### Chemical Synthetic Methods for Preparing Unique Tags and Uniquely Tagged Probes

Unique tags and uniquely tagged probes can be generated by in situ synthesis of parameter signatures or specific chemical tags. Methods are known in the art to use light to drive synthesis of oligonucleotides, for example. A mixed population of physical entities with different specific signatures can all be chemically primed for oligonucleotide synthesis. The entities can then be attached to a surface and the location of each identity determined by optical scanning and stored in a computer. Then one nucleotide can be introduced in the liquid solution in contact with the physical entities. A targeted light source can then be used to catalyze the chemical reaction of that nucleotide on the physical entities targeted. The first nucleotide is then removed and a second nucleotide is introduced. The light source is then used to induce the addition of the nucleotide in the locations where it is needed. This process can then be repeated until oligonucleotides of a specified length and sequence have been synthesized on each of the physical entities.

As a specific example, it is desired to produce three different oligonucleotides of sequence, ATGC, AGCT, and GCTA, where A, T, C, G are the standard abbreviations for the four bases found in DNA. Beads of three sizes, for example, 1, 3 and 10 microns, are prepared. It is desired to associate the one micron bead to ATGC, the 3 micron bead to ACGT and the 10 micron bead to GCTA. The three beads sizes are mixed in equal numbers and placed in a particular location and attached to a surface. A nucleotide with base A is then introduced to the solution. A laser is used to drive the synthesis of A on the one and three micron beads. The signature status of the beads is now 1-A, 3-A, and 10 is blank. Nucleotide G is then introduced into the solution and synthesis is induced on the three and ten micron beads. The signature status of the beads is now I-A, 3-AG, and 10-G. This process is then repeated until a signature status of 1-ATGC, 3-AGCT, and 10-GCTA, is obtained. The sequence of nucleotide additions into the solution can be optimized using standard methods based on the sequence of the tags. This procedure in principle is completely scalable for any number of physical entities with unique parameter signatures and length of the DNA sequence desired.

Similarly, in situ synthesis of unique parameter signatures can be obtained. A mixture of beads of different sizes that have been chemically primed for conjugation can be placed on a surface, attached to the surface and optically scanned to determine their locations. Particles or chemical compounds that will be used to define the unique parameter signature are then introduced. For example, quantum dots of multiple emission wavelengths, or nano-tubes of particular specifications can be used to tag physical entities.

In a specific example, it is desired to obtain three color (Red, Green, and Blue, or R, G and B respectively) tagging onto beads of three different sizes (1, 3 and 10 micron), and in addition to grade the colors into three intensities (1, 2, 3 relative units). First, a green nanodot is introduced into the solution above the surface where the mixture beads of three sizes is located. The laser is then used (by varying intensity or the number of pulses aimed at each bead) to produce beads of each size with three green intensities. Symbolically, the reaction has produced 1-G, 1-GG, 1-GGG, 3-G, 3-GG, 3-GGG, 1 O-G, 10-GG, 1 O-GGG where the number is the bead size and the number of Gs is the intensity. If the correct intensities are not achieved for a particular bead, that bead can be destroyed to insure that there are no overlapping parameter signatures. A red nanodot then can be introduced and the procedure repeated. The result for the 1 micron bead will be nine combinations of 1-GR, 1-GGR, 1-GGGR, 1-GRR, 1-GGRR, 1-GGGRR, 1-GRRR, 1-GGRRR, and 1-GGGRRR with similar results for the 3 and 10 micron beads to generate a total of 27 unique parameter signatures. The addition of the blue bead then gives three variations of each of these 27 combinations. For example, the 1-GR signature now be comes three variants of 1-GRB, 1-GRBB, and 1-GRBBB. Thus a total of 81 unique signatures will be generated (81=34 given that there are four parameters, size, G, R, and B, at three levels).

### EXAMPLE V

### Detection of Single Nucleotide Polymorphisms (SNPs)

This example describes the use of a population of uniquely tagged probes to simultaneously detect thousands of nucleic acid sequences in a single assay.

To generate an population of uniquely tagged probes to detect 100,000 analytes, a set of physical entities that each have 5 features (such as size, color, fluorescence, etc) where each feature has a grading of 10 increments, can be generated. For example, the size of the beads can be 2, 4, 6, 8, 10, 12, 14, 16, 18, and 20 micron. All features can be determined simultaneously. A population of beads, each having a unique specific signature can be produced, for example, as described in Example IV.

A unique complementary nucleic acid probe that hybridizes to each polymorphism to be detected is then attached to each homogeneous population of uniquely tagged physical entities. This results in about 100,000 beads, each having a unique specific signature, where each bead is a probe for a unique nucleotide sequence. More than 1 copy of each unique bead is used in the assay as dictated by experimental statistical design to obtain a meaningful number of replicates of each polymorphic assay to be performed. For example, if such number of required beads of each unique parameter signature is 10 then a total of about 1,000,000 beads can be prepared.

The DNA from the individual to be genotyped is processed using methods known in the art and conjugated using a single fluorochrome, such as Cy3 or Cy5. This DNA preparation is then exposed to the population of 100,000 beads for a sufficient time and under conditions that enable hybridization to take place between complementary DNA strands. Thus a bead with a unique specific signature is associated with a hybridized complementary sequence found in the original DNA sample. Upon scanning the population, the association between the uniquely tagged probes and the fluorescently tagged DNA fragments derived from the individual's DNA sample can be observed. A 1,000,000-marker genotype of the individual is then obtained.

Alternatively, a population of uniquely tagged probes prepared using uniquely tags having common specific signatures can be used. The specific signatures of each probes are differentiated by separating uniquely tagged probes having the same specific signature in different physical locations, such as wells of a multi-well plates.

### EXAMPLE VI

### DNA Sequencing

This example shows the use of a large population of uniquely tagged probes to determine a nucleic acid sequence. With 4 basic bases on DNA, a sequence of n-bases (also called an n-mer) can generate 4ⁿ unique sequences. Using 10 bases (a 10-mer) as an example, the number of possible sequences is 4¹⁰=1,048,576. Thus if 1,048,576 uniquely tagged probes, each with a unique 10-mer, are prepared, the sequence of any 10-mer can be determined. A linker between the physical entity and the 10-mer can be created for optimal hybridization conditions as known in the art.

Similarly, a 20-mer sequence is a series of 10 unique overlapping 10-mer sequences offset by a number of base pairs, for example 1, 2, 3 or more base pairs. In an example in which a 1 base pair offset is employed, one 10-mer probe uniquely hybridizes to base pairs 1 through 10 on the 20-mer, another 10-mer will identify base pairs 2 through 11 and so forth. Thus any DNA sequence can be determined this way in principle by proper assembly of the overlapping sequences, such as the 10-mers described in the above example, as is known in the art.

If a longer sequence is desired for specific associations this procedure can be scaled up. For example, if the probing sequence needs to be a 15-mer then 4¹⁵=1,073,741,824 uniquely tagged probes are needed. Thus sequencing of any DNA molecule is achievable if 1,073,741,824 uniquely tagged probes are prepared. Alternatively, 1,073,742 uniquely tagged physical entities can be prepared such that 1000 probes share the same uniquely identifying tag. In this case, the assay could be multiplexed in 1000 wells. The number of assays performed by can be increased by increasing the number of wells to match the number of unique assays, or association events, and the number of uniquely tagged physical entities available. Thus, if one can scan a 1536 well plate in 3 minutes, in one hour 30,520 wells can be scanned, which with a 1000 unique bead signatures gives over 30 million unique assays.

The methods for DNA sequencing using uniquely tagged probes prepared using the methods of the invention can involve a variety of hybridization methods and conditions, including probe hybridization temperature cycling. Temperature cycling can be used to increase the accuracy of methods for sequencing by probe hybridization. For example, hybridization of uniquely tagged probes can be performed below the melting temperature of the uniquely tagged probe, and the temperature can be cyclically increased and decreased around the melting temperature of the uniquely tagged probe to reduce any non-specific binding.

### EXAMPLE VII

### Expression Profiling

Considerable interest has developed in recent years in determining all the messenger RNA (mRNA) molecules present in a cell at a given time. This procedure is known as mRNA expression profiling. Microarrays (BrownBotstein), photolithographic methods (Affy) and micro-electronic mirrors (ret) have been used to array in a spatially regular pattern complementary mRNA probes on a surface. In this fashion a unique hybridization event can be determined in a predetermined location.

Following the sequencing and SNP example given above, unique nucleic acid sequences complementary for a mRNA molecule corresponding to an expressed segment of a genomic sequence can be linked to a physical entity, such as a bead. The mRNA is labeled with methods described in the art so that the association can be detected.

### EXAMPLE VIII

### Antibody Screening

The antibody repertoire of the human immune system is estimated to be able to detect about a billion different chemical structures (epitopes). Thus a library of a billion antibodies should be able to detect any chemical structure of interest. As described above, a billion unique parameter signatures can be attached to a billion different physical entities. A pure population of one such physical entity can be conjugated to a target specific antibody, such as a monoclonal antibody. Conjugation can be achieved using methods well known to those skilled in the art. A billion such beads, each with a unique parameter signature, can be made each containing a unique antibody that can bind a unique epitope. This population of billion beads can then be brought into contact with a ligand (or an antigen) for which we want to find a selective antibody. The ligand is labeled with a detectable probe, such as a fluorochrome as known in the art and allowed to bind to the antibodies on beads. Then the entire population of billion different bead can be screened for the association of a bead and the ligand. Alternatively, multiplexing through can be performed when two or more different antibodies are labeled with the same uniquely identifying tag by placing antibodies labeled with the same uniquely identifying tag at physically separate locations. It is expected that more than one association is found and thus a series of candidate antibodies for binding to the ligand can be identified. These selected antibodies can then be further characterized for their binding constants using known methods in the art.

### EXAMPLE IX

### Assays Performed in Micelles

Artificial membranes, such as micelles, can be tagged with a uniquely identifying tag using the methods of the invention. Artificial membranes are useful for enhancing stability of biological molecules and allow associations between members of molecular complexes. For example, by co-localizing the molecules in close proximity and by retaining solubility of molecules in the absence of detergents, signaling complexes can be functionally reconstituted. A variety to multi-component biochemical processes can be reconstituted in artificial membranes, such as micelles. G-protein coupled signal transduction is one example of a successfully reconstituted multi-component system.

For use in the methods of the invention, a reconstituted system, such as a micelle, is prepared and purified to meet physical property specifications similar to those described above in reference to a homogeneous population of beads. For example, an external energy source can be used to destroy all micelles that do not meet a size specification. Another selection criteria can be the presence or absence of a detectable component in the micelle. For example, one of the proteins can be tagged with an optically detectable probe, such as a fluorescent dye or an RLS particle. The presence of this tagged component can then be detected and used as an inclusion criterion.

A homogeneous population of the micelles is then spatially separated, for example through the use of multi-well plates, and a different molecule added into each location to determine the response of the reconstituted signaling process to that particular chemical entity. This response can include the kinetics of the function of the reconstituted system. In this manner, a large number of miniaturized assays to probe reactions to different compounds can be performed.

Alternatively, a defined diverse population of micelles is prepared. A component in the reconstituted system can have one or more different genetic variants, for example multiple variants of a component of a G-protein coupled signaling system. Each of the variants is uniquely tagged, such that each micelle containing a different variant is distinguishable based on a unique parameter signature and is locatable within a population. A compound is introduced to this heterogeneous preparation of micelles, and a response of each different variant of the reconstituted system determined simultaneously. A heterogeneous population of reconstituted processes can be placed in many physically separated locations, where in each location a different compound is found to stimulate the reconstructed process. Thus, each assay simultaneously measures the response of all the variants of a reconstituted biochemical process to a single compound in a multiplexed fashion.

Although the invention has been described with reference to the disclosed embodiments, those skilled in the art will readily appreciate that the specific experiments detailed are only illustrative of the invention. It should be understood that various modifications can be made without departing from the spirit of the invention.

What is claimed is:

1. A method for selecting a population of non-cellular physical entities, comprising applying energy to one or more non-cellular physical entities having selected parameter signatures, each physical entity located at specific coordinates in a domain and contained within a population of physical entities, thereby altering a property of the one or more physical entities, wherein the alteration renders the one or more physical entities separable from other members of the population of physical entities.

2. The method of claim 1, wherein a non-cellular physical entity having a selected parameter signature has an undesired parameter signature.

3. The method of claim 2, wherein the applied energy destroys the one or more physical entities.

4. The method of claim 1, wherein a non-cellular physical entity having a selected parameter signature has a desired parameter signature.

5. The method of claim 2 or 4, wherein the applied energy induces attachment of the one or more physical entities to the domain.

6. The method of claim 1, further comprising separating the one or more altered physical entities from the population of physical entities.

7. The method of claim 1 or 6, wherein energy is applied to 10 or more non-cellular physical entities having selected parameter signatures.

8. The method of claim 1 or 6, wherein energy is applied to 100 or more non-cellular physical entities having selected parameter signatures.

9. The method of claim 1 or 6, wherein energy is applied to 1000 or more non-cellular physical entities.

10. The method of claim 1, wherein a population of non-cellular physical entities each having a distinct signature parameter is selected.

11. The method of claim 1, wherein a population of non-cellular physical entities having homogeneous signature parameters is selected.

12. The method of claim 1, wherein a parameter signature of a physical entity is determined by one or more characteristics selected from the group consisting of size, shape, color, fluorescence emission and fluorescence absorption.

13. The method of claim 1, wherein the coordinates of a targeted physical entity are determined by a method comprising, (a) capturing an image of the population of physical entities; (b) identifying a targeted physical entity in the image and (c) assigning coordinates to the targeted physical entity.

14. The method of claim 1, wherein the coordinates of a targeted physical entity are determined by a method comprising, (a) obtaining a plurality of nonidentical two-dimensional sectional representations a domain containing physical entities, in which the targeted physical entity is discernable in at least one of the sectional representations; (b) combining the plurality of sectional two-dimensional representations to produce a three-dimensional representation of the domain; (c) locating the targeted physical entity in three dimensions based on the three-dimensional representation, and (d) assigning coordinates to the targeted physical entity.

15. The method of claim 13 or 14, further comprising indexing the coordinates.

16. The method of claim 1, wherein the energy is applied from a controlled energy source.

17. The method of claim 1, wherein the energy is applied from two or more controlled energy sources.

18. The method of claim 16 or 17, wherein the controlled energy source is a laser.

19. A method for preparing a population of uniquely tagged non-cellular physical entities, comprising:
(a) contacting a population of non-cellular physical entities with a chemical agent;
(b) applying energy to one or more targeted physical entities, the energy capable of inducing attachment of the chemical agent to a targeted physical entity;
(c) separating unattached chemical agent from chemical agent attached to the one or more targeted physical entities, and
(d) repeating steps (a), (b) and (c) using a distinct chemical agent to produce a population of uniquely tagged non-cellular physical entities.

20. The method of claim 19, wherein step (d) further comprises repeating steps (a), (b) and (c) 10 or more times.

21. The method of claim 19, wherein step (d) further comprises repeating steps (a), (b) and (c) 100 or more times.

22. The method of claim 19, wherein step (d) further comprises repeating steps (a), (b) and (c) 1000 or more times.

23. The method of claim 19, wherein the chemical agent is a chemical agent selected from the group consisting of detectable chemical agent, polynucleotide, nucleotide, polypeptide and amino acid.

24. The method of claim 19, wherein each targeted physical entity in the population of non-cellular physical entities has a distinct specific signature.

25. The method of claim 24, wherein a specific signature of a physical entity is defined by one or more characteristics selected from the group consisting of size, shape, color, fluorescence emission and fluorescence absorption.

26. The method of claim 19, wherein coordinates of a targeted physical entity are determined by a method comprising, (a) capturing an image of the population of physical entities; (b) identifying a physical entity in the image and (c) assigning coordinates to the physical entity.

27. The method of claim 19, wherein coordinates of a targeted physical entity are determined by a method comprising, (a) obtaining a plurality of nonidentical two-dimensional sectional representations the domain, in which the physical entity is discernable in at least one of the sectional representations; (b) combining the plurality of sectional two-dimensional representations to produce a three-dimensional representation of the domain; (c) locating the physical entity in three dimensions based on the three-dimensional representation, and (d) assigning coordinates to the physical entity.

28. The method of claim 26 or 27, further comprising indexing the coordinates.

29. The method of claim 19, wherein the one or more physical entities are attached to a domain.

30. The method of claim 19, wherein step (a) further comprises attaching one or more physical entities to a specific location on a domain.

31. The method of claim 20, wherein the one or more physical entities are attached to a domain by applying energy to the physical entities.

32. The method of claim 19, wherein the energy is applied from a controlled energy source.

33. The method of claim 31, wherein the energy is applied from two or more controlled energy sources.

34. The method of claim 32 or 33, wherein the controlled energy source is a laser.

35. A method for preparing a population of uniquely tagged non-cellular physical entities, comprising:
(a) associating a population of physical entities with two or more reaction spaces on a domain, each reaction space containing a different chemical agent, and
(b) applying energy to a targeted physical entity in each of one or more reaction spaces, the energy capable of inducing attachment of a chemical agent to the physical entity, thereby generating a population of uniquely tagged physical entities.

36. The method of claim 35, further comprising removing unattached chemical agent from chemical agent attached to the targeted physical entity.

37. The method of claim 36, further comprising repeating steps (a) and (b) one or more time, each time using a distinct chemical agent in each reaction space, the chemical agent capable of attachment to a selected physical entity or chemical agent attached to a selected physical entity.

38. The method of claim 35, wherein each member of the population of non-cellular physical entities has a distinct specific signature.

39. The method of claim 35, wherein a specific signature of a physical entity is determined by one or more characteristics selected from the group consisting of size, shape, color, fluorescence emission and fluorescence absorption.

40. The method of claim 35, wherein the chemical agent is selected from the group consisting of detectable chemical agent, polynucleotide, nucleotide, polypeptide and amino acid.

41. The method of claim 35, wherein the coordinates of a targeted physical entity are determined by a method comprising, (a) capturing an image of the population of physical entities; (b) identifying a targeted physical entity in the image and (c) assigning coordinates to the targeted physical entity.

42. The method of claim 35, wherein the coordinates of a targeted physical entity are determined by a method comprising, (a) obtaining a plurality of nonidentical two-dimensional sectional representations of the domain, in which the targeted physical entity is discernable in at least one of the sectional representations; (b) combining the plurality of sectional two-dimensional representations to produce a three-dimensional representation of the domain; (c) locating the targeted physical entity in three dimensions based on the three-dimensional representation, and (d) assigning coordinates to the targeted physical entity.

43. The method of claim 41 or 42, further comprising indexing the coordinates.

44. The method of claim 35, wherein the energy is applied from a controlled energy source.

45. The method of claim 44, wherein the energy is applied from two or more controlled energy sources.

46. The method of claim 44 or 45, wherein the controlled energy source is a laser.

47. A method for preparing a population of uniquely tagged probes, comprising:
(a) contacting a population of uniquely tagged non-cellular physical entities with a target moiety;
(b) applying energy to one or more targeted uniquely tagged physical entities, the energy capable of inducing attachment of the target moiety to a targeted physical entity;
(c) separating unattached target moiety from target moiety attached to the one or more targeted uniquely tagged physical entities, and
(d) repeating steps (a), (b) and (c) using a distinct target moiety to label another member of the population of physical entities, thereby generating a population of uniquely tagged probes.

48. The method of claim 47, wherein the target moiety is selected from the group consisting of polynucleotide, oligonucleotide, polypeptide, antibody, antigen, ligand and receptor.

49. A method for preparing a population of uniquely tagged probes, comprising:
(a) associating a population of uniquely tagged physical entities with two or more reaction spaces on a domain, each reaction space containing a different target moiety, and
(b) applying energy to a targeted uniquely tagged physical entity in each of one or more reaction spaces, the energy capable of inducing attachment of a target moiety to the physical entity, thereby generating a population of uniquely tagged probes.

50. The method of claim 49, wherein the target moiety is selected from the group consisting of polynucleotide, oligonucleotide, polypeptide, antibody, antigen, ligand and receptor.

51. A method for simultaneously detecting a plurality of analytes, comprising:
(a) contacting a population of uniquely tagged probes of claim 47 or 49 with a sample, and
(b) detecting an interaction between one or more uniquely tagged probes and a cognate binding partner.
